(19) United States
(12) Patent Application Publication
(10) Pub. No.: US 2002/0025937 A1
(43) Pub. Date: Feb. 28, 2002

(54) 9-OXIME ERYTHROMYCIN DERIVATIVES

(76) Inventor: YONG-JIN WU, MADISON, CT (US)

Correspondence Address: PAUL H GINSBURG, PFIZER INC, 235 EAST 42ND STREET, 20TH FLOOR, NEW YORK, NY, 10017-5755, US

(21) Appl. No.: 09/445,780

(22) PCT Filed: May 15, 1998
(86) PCT No.: PCT/IB98/00741

(51) Int. Cl. C07H 17/08; A61K 31/70; A01N 43/04

(52) U.S. Cl. 514/29; 536/7.4

(57) ABSTRACT

The invention relates to compounds of the formula I

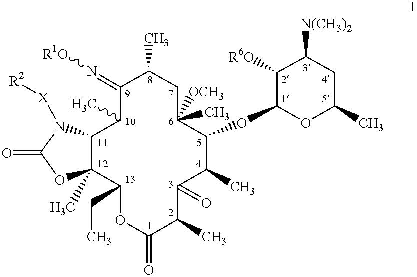

and to pharmaceutically acceptable salts thereof, wherein R¹, R², R⁶ and X are as defined herein. The invention also relates to pharmaceutical compositions containing the compounds of formula I, methods of using said compounds of formula I in the treatment of bacterial and protozoa infections, and methods of preparing said compounds of formula I.

### BACKGROUND OF THE INVENTION

This invention relates to novel 3-keto-9-oxime-11,12-carbazate or carbamate derivatives of 6-O-methyl-erythromycin A. The compounds of this invention are useful as antibiotic agents in mammals, including man, as well as in fish and birds. The compounds of the present invention are broad-spectrum macrolide antibiotics that are effective against infections caused by certain gram-positive and gram-negative bacteria as well as protozoa.

### SUMMARY OF THE INVENTION

The present invention relates to compounds of the formula

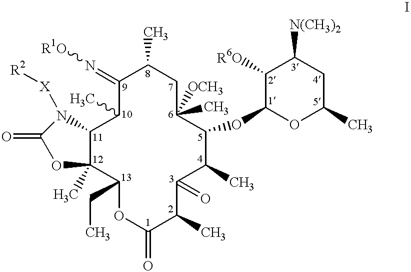

and to pharmaceutically acceptable salts thereof, wherein:

X is —CR⁷R⁸— or —NR⁷—;

or X is taken together with R² to form —N═CR⁴R⁵;

or X and R² are taken together to form a heterocyclic ring of the formula XVI:

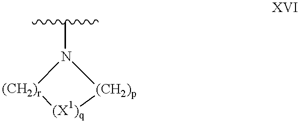

wherein in said ring of formula XVI, r and p are each independently an integer ranging from 1 to 3, q is 0 or 1, and X¹ is —CH₂—, O, S, —C(O)—, —C(S)—, —SO₂—, —CH═CH—, —CH(OH)CH(OH)—, or —NH—; and wherein the (CH₂)ᵣ and (CH₂)ₚ portions of said ring of formula XVI are optionally substituted by 1 to 4 substituents, and the nitrogen atom where X¹ is —NH— is optionally substituted by 1 substituent, said optional substituents being independently selected from the group consisting of —C(O)O(C₁-C₁₀ alkyl), C₁-C₁₀ alkoxy, C₁-C₁₀ alkanoyl, halo, nitro, cyano, 5-10 membered heterocyclyl, C₁-C₁₀ alkyl, —NR⁷R⁸, C₆-C₁₀ aryl, —S(O)ₙ(C₁-C₁₀ alkyl) wherein n is an integer ranging from 0 to 2, and —SO₂NR⁷R⁸;

R¹ is H or C₁-C₁₀ alkyl, wherein 1 to 3 carbons of said alkyl are optionally replaced by a heteroatom selected from O, S and N, and said alkyl is optionally substituted by 1 to 3 substituents independently selected from the group consisting of —C(O)O(C₁-C₁₀ alkyl), C₁-C₁₀ alkoxy, C₁-C₁₀ alkanoyl, halo, nitro, cyano, 5-10 membered heterocyclyl, C₁-C₁₀ alkyl, —NR⁷R⁸, C₆-C₁₀ aryl, —S(O)ₙ(C₁-C₁₀ alkyl) wherein n is an integer ranging from 0 to 2, and —SO₂NR⁷R⁸;

R² is (i) H, R⁴, —C(O)R⁴, —C(O)OR⁴ or —(CR⁷R⁸)ₘR³ when X is —NR⁷—, or (ii) H, R⁴, or —(CR⁷R⁸)ₘR³ when X is —CR⁷R⁸—, wherein for both (i) and (ii) m is an integer ranging from 0 to 6 and both R⁷ and R⁸ may vary for each iteration where m is greater than 1;

each R³ is independently C₆-C₁₀ aryl or 5-10 membered heterocyclyl, wherein said aryl and heterocyclyl groups are optionally substituted by 1 to 3 substituents independently selected from the group consisting of —C(O)O(C₁-C₁₀ alkyl), C₁-C₁₀ alkoxy, C₁-C₁₀ alkanoyl, halo, nitro, cyano, 5-10 membered heterocyclyl, C₆-C₁₀ aryl, C₁-C₁₀ alkyl, —NR⁷R⁸, —S(O)ₙ(C₁-C₁₀ alkyl) wherein n is an integer ranging from 0 to 2, and —SO₂NR⁷R⁸; and,

each R⁴ and R⁵ is independently selected from H and C₁-C₁₂ alkyl wherein one or two carbons of said alkyl are optionally replaced by a heteroatom selected from O, S and N, and wherein said alkyl is optionally substituted by 1 to 3 substituents independently selected from the group consisting of —C(O)O(C₁-C₁₀ alkyl), C₁-C₁₀ alkoxy, C₁-C₁₀ alkanoyl, halo, nitro, cyano, C₁-C₁₀ alkyl, —NR⁷R⁸, C₆-C₁₀ aryl, 5-10 membered heterocyclyl, —S(O)ₙ(C₁-C₁₀ alkyl) wherein n is an integer ranging from 0 to 2, and —SO₂NR⁷R⁸;

R⁶ is H, —C(O)R³ or C₁-C₁₈ alkanoyl, wherein in the alkyl portion of said alkanoyl one or two carbons optionally may be replaced by a heteroatom selected from O, S and N; and,

each R⁷ and R⁸ is independently H or C₁-C₆ alkyl.

More specific embodiments of this invention include compounds of formula I wherein R⁶ is H.

Other more specific embodiments of this invention include compounds of formula I wherein X is —NH—.

Other more specific embodiments of this invention include compounds of formula I wherein R¹ is H, benzyl or C₁-C₃ alkyl or —CH₂O(CH₂)₂OCH₃.

Other more specific embodiments of this invention include compounds of formula I wherein R² is —(CH₂)ₘR³ wherein m is an integer ranging from 0 to 6 and R³ is 5-10 membered heterocyclyl or C₆-C₁₀ aryl. Specific embodiments of R³ include quinolin-4-yl, 4-phenyl-imidazol-1-yl, imidazo(4,5-b)pyridin-3-yl, 4-pyridin-3-ylimidazol-1-yl and pyridin-3-yl.

Examples of preferred compounds of this invention include:

9-Deoxo-11-deoxy-5-O-desosaminyl-11-(3-(4-pyridin-3-yl-imidazol-1-yl)-propyl)hydrazo-9-hydroxyimino-6-O-methyl-3-oxoerythronolide A, 11,12-carbamate;

9-Deoxo-11-deoxy-5-O-desosaminyl-11-(3-(4-pyridin-3-yl-imidazol-1-yl)-propyl)hydrazo-9-methoxyimino-6-O-methyl-3-oxoerythronolide A, 11,12-carbamate;

9-Deoxo-11-deoxy-5-O-desosaminyl-11-(3-(imidazo(4,5-b)pyridin-3-yl)-propyl)hydrazo-9-hydroxyimino-6-O-methyl-3-oxoerythronolide A, 11,12-carbamate;

9-Deoxo-11-deoxy-5-O-desosaminyl-11-(3-(imidazo(4,5-b)pyridin-3-yl)-propyl)hydrazo-9-methoxyimino-6-O-methyl-3-oxoerythronolide A, 11,12-carbamate;

9-Deoxo-11-deoxy-5-O-desosaminyl-11-(3-quinolin-4-yl-propyl)hydrazo-9-hydroxyimino-6-O-methyl-3-oxoerythronolide A, 11,12-carbamate;

9-Deoxo-11-deoxy-5-O-desosaminyl-11-(3-quinolin-4-yl-propyl)hydrazo-9-methoxyimino-6-O-methyl-3-oxoerythronolide A, 11,12-carbamate;

9-Deoxo-11-deoxy-5-O-desosaminyl-11-(3-(7-methoxy-quinolin-4-yl)-propyl))hydrazo-9-hydroxyimino-6-O-methyl-3-oxoerythronolide A, 11,12-carbamate;

9-Deoxo-11-deoxy-5-O-desosaminyl-11-(3-(7-methoxy-quinolin-4-yl)-propyl))hydrazo-9-methoxyimino-6-O-methyl-3-oxoerythronolide A, 11,12-carbamate

9-Deoxo-11-deoxy-5-O-desosaminyl-11-(3-quinolin-4-yl-propylidene)hydrazo-9-hydroxyimino-6-O-methyl-3-oxoerythronolide A, 11,12-carbamate;

9-Deoxo-11-deoxy-5-O-desosaminyl-11-(3-quinolinlyl-propylidene)hydrazo-9-methoxyimino-6-O-methyl-3-oxoerythronolide A, 11,12-carbamate;

9-Deoxo-11-deoxy-5-O-desosaminyl-11-(3-quinolin-4-yl-propyl)hydrazo-9-benzoxyimino-6-O-methyl-3-oxoerythronolide A, 11,12-carbamate;

19-Deoxo-1-deoxy-5-O-desosaminyl-11-(3-benzoimidazol-1-yl-propyl)hydrazo-9-hydroxyimino-6-O-methyl-3-oxoerythronolide A, 11,12-carbamate;

19-Deoxo-1-deoxy-5-O-desosaminyl-11-(3-benzoimidazol-1-yl-propyl)hydrazo-9-methoxyimino -6O-methyl-3-oxoerythronolide A, 11,12-carbamate;

9-Deoxo-11-deoxy-5-O-desosaminyl-ll-(3-indol-1-yl-propyl)hydrazo-9-hydroxyimino-6-O-methyl-3-oxoerythronolide A, 11,12-carbamate;

9-Deoxo-11-deoxy-5-O-desosaminyl-11-(3-indol-1-yl-propyl)hydrazo-9-methoxyimino-6-O-methyl-3-oxoerythronolide A, 11,12-carbamate;

9-Deoxo-11-deoxy-5-O-desosaminyl-ll-(3-indazol-1-yl-propyl)hydrazo-9-hydroxyimino-6-O-methyl-3-oxoerythronolide A, 11,12-carbamate;

9-Deoxo-11-deoxy-5-O-desosaminyl-11-(3-indazol-1-yl-propyl)hydrazo-9-methoxyimino-6-O-methyl-3-oxoerythronolide A, 11,12-carbamate;

9-Deoxo-11-deoxy-5-O-desosaminyl-11-(3-carbazol-1-yl-propyl)hydrazo-9-hydroxyimino-6-O-methyl-3-oxoerythronolide A, 11,12-carbamate;

9-Deoxo-11-deoxy-5-O-desosaminyl-11-(3-carbazol-1-yl-propyl)hydrazo-9-methoxyimino-6-O-methyl-3-oxoerythronolide A, 11,12-carbamate;

9-Deoxo-11-deoxy-5-O-desosaminyl-11-(3-(5-phenyl-1H-pyrrol-2-yl)-propyl)hydrazo-9-hydroxyimino-6-O-methyl-3-oxoerythronolide A, 11,12-carbamate;

9-Deoxo-11-deoxy-5-O-desosaminyl-11-(3-(5-phenyl-1H-pyrrol-2-yl)-propyl)hydrazo-9-methoxyimino-6-O-methyl-3-oxoerythronolide A, 11,12-carbamate;

9-Deoxo-11-deoxy-5-O-desosaminyl-11-(3-(4-phenyl-imidazol-1-yl)-propyl)hydrazo-9-hydroxyimino-6-O-methyl-3-oxoerythronolide A, 11,12-carbamate;

9-Deoxo-11-deoxy-5-O-desosaminyl-11-(3-(4-phenyl-imidazol-1-yl)-propyl)hydrazo-9-methoxyimino-6-O-methyl-3-oxoerythronolide A, 11,12-carbamate;

9-Deoxo-11-deoxy-5-O-desosaminyl-11-(3-(3-(4-chlorophenyl)-(1,2,4)oxadizol-5-yl)-propyl)hydrazo-9-hydroxyimino-6-O-methyl-3-oxoerythronolide A, 11,12-carbamate;

9-Deoxo-11-deoxy-5-O-desosaminyl-11-(3-(3-(4-chlorophenyl)-(1,2,4)oxadizol-5-yl)-propyl)hydrazo-9-methoxyimino-6-O-methyl-3-oxoerythronolide A, 11,12-carbamate;

19-Deoxo-1-deoxy-5-O-desosaminyl-11-(3-(3-(4-methoxyphenyl)-(1,2,4)oxadizol-5-yl)-propyl)hydrazo-9-hydroxyimino-6-O-methyl-3-oxoerythronolide A, 11,12-carbamate;

19-Deoxo-1-deoxy-5-O-desosaminyl-11-(3-(3-(4-methoxyphenyl)-(1,2,4)oxadizol-5-yl)-propyl)hydrazo-9-methoxyimino-6-O-methyl-3-oxoerythronolide A, 11,12-carbamate;

9-Deoxo-11-deoxy-5-O-desosaminyl-11-(3-(3-(4-pyridin-4-yl)-(1,2,4)oxadizol-5-yl)-propyl)hydrazo-9-hydroxyimino-6-O-methyl-3-oxoerythronolide A, 11,12-carbamate;

9-Deoxo-11-deoxy-5-O-desosaminyl-11-(3-(3-(4-pyridin-4-yl)-(1,2,4)oxadizol-5-yl)-propyl)hydrazo-9-methoxyimino-6-O-methyl-3-oxoerythronolide A, 11,12-carbamate;

9-Deoxo-11-deoxy-5-O-desosaminyl-11-(3-naphthalen-1-yl-propyl)hydrazo-9-hydroxyimino-6-O-methyl-3-oxoerythronolide A, 11,12-carbamate;

9-Deoxo-11-deoxy-5-O-desosaminyl-11-(3-naphthalen-1-yl-propyl)hydrazo-9-methoxyimino-6-O-methyl-3-oxoerythronolide A, 11,12-carbamate;

9-Deoxo-11-deoxy-5-O-desosaminyl-11-(3-benzotrizol-1-yl-propyl)hydrazo-9-hydroxyimino-6-O-methyl-3-oxoerythronolide A, 11,12-carbamate;

9-Deoxo-11-deoxy-5-O-desosaminyl-11-(3-benzotrizol-1-yl-propyl)hydrazo-9-methoxyimino-6-O-methyl-3-oxoerythronolide A, 11,12-carbamate;

9-Deoxo-11-deoxy-5-O-desosaminyl-11-(3-benzotrizol-2-yl-propyl)hydrazo-9-hydroxyimino-6-O-methyl-3-oxoerythronolide A, 11,12-carbamate;

9-Deoxo-11-deoxy-5-O-desosaminyl-11-(3-benzotrizol-2-yl-propyl)hydrazo-9-methoxyimino-6-O-methyl-3-oxoerythronolide A, 11,12-carbamate;

9-Deoxo-11-deoxy-5-O-desosaminyl-11-(3-(1H-indol-3-yl)-propyl)hydrazo-9-hydroxyimino-6-O-methyl-3-oxoerythronolide A, 11,12-carbamate;

9-Deoxo-11-deoxy-5-O-desosaminyl-11-(3-(1H-indol-3-yl)-propyl)hydrazo-9-methoxyimino-6-O-methyl-3-oxoerythronolide A, 11,12-carbamate;

9-Deoxo-11-deoxy-5-O-desosaminyl-11-(3-pyridin-4-yl-propyl)hydrazo-9-hydroxyimino-6-O-methyl-3-oxoerythronolide A, 11,12-carbamate;

9-Deoxo-11-deoxy-5-O-desosaminyl-11-(3-pyridin-4-yl-propyl)hydrazo-9-methoxyimino-6-O-methyl-3-oxoerythronolide A, 11,12-carbamate;

9-Deoxo-11-deoxy-5-O-desosaminyl-11-(3-pyridin-3-yl-propyl)hydrazo-9-hydroxyimino-6-O-methyl-3-oxoerythronolide A, 11,12-carbamate;

9-Deoxo-11-deoxy-5-O-desosaminyl-11-(3-pyridin-3-yl-propyl)hydrazo-9-methoxyimino-6-O-methyl-3-oxoerythronolide A, 11,12-carbamate;

9-Deoxo-11-deoxy-5-O-desosaminyl-11-(3-(pyridin-2-yl-propyl)hydrazo-9-hydroxyimino-6-O-methyl-3-oxoerythronolide A, 11,12-carbamate;

9-Deoxo-11-deoxy-5-O-desosaminyl-11-(3-(pyridin-2-yl-propyl)hydrazo-9-methoxyimino-6-O-methyl-3-oxoerythronolide A, 11,12-carbamate;

9-Deoxo-11-deoxy-5-O-desosaminyl-11-(3-phenylpropyl)hydrazo-9-hydroxyimino-6-O-methyl-3-oxoerythronolide A, 11,12-carbamate;

9-Deoxo-11-deoxy-5-O-desosaminyl-11-(3-phenylpropyl)hydrazo-9-methoxyimino-6-O-methyl-3-oxoerythronolide A, 11,12-carbamate;

9-Deoxo-11-deoxy-5-O-desosaminyl-11-bis-(3-phenylpropyl)hydrazo-9-hydroxyimino-6-O-methyl-3-oxoerythronolide A, 11,12carbamate;

9-Deoxo-11-deoxy-5-O-desosaminyl-11-bis-(3-phenylpropyl)hydrazo-9-methoxyimino-6-O-methyl-3-oxoerythronolide A, 11,12 carbamate;

9-Deoxo-11-deoxy-5-O-desosaminyl-11-(3-(4-methoxyphenyl)-propyl)hydrazo-9-hydroxyimino-6-O-methyl-3-oxoerythronolide A, 11,12-carbamate;

9-Deoxo-11-deoxy-5-O-desosaminyl-11-(3-(4-methoxyphenyl)-propyl)hydrazo-9-methoxyimino-6-O-methyl-3-oxoerythronolide A, 11,12-carbamate;

9-Deoxo-11-deoxy-5-O-desosaminyl-11-(3-(3-methoxyphenyl)-propyl)hydrazo-9-hydroxyimino-6-O-methyl-3-oxoerythronolide A, 11,12-carbamate;

9-Deoxo-11-deoxy-5-O-desosaminyl-11-(3-(3-methoxyphenyl)-propyl)hydrazo-9-methoxyimino-6-O-methyl-3-oxoerythronolide A, 11,12-carbamate;

9-Deoxo-11-deoxy-5-O-desosaminyl-11-(3-(2-methoxyphenyl)-propyl)hydrazo-9-hydroxyimino-6-O-methyl-3-oxoerythronolide A, 11,12-carbamate;

9-Deoxo-11-deoxy-5-O-desosaminyl-11-(3-(2-methoxyphenyl)-propyl)hydrazo-9-methoxyimino-6-O-methyl-3-oxoerythronolide A, 11,12-carbamate;

9-Deoxo-11-deoxy-5-O-desosaminyl-11-(3-(4-hydroxyphenyl)-propyl)hydrazo-9-hydroxyimino-6-O-methyl-3-oxoerythronolide A, 11,12-carbamate;

9-Deoxo-11-deoxy-5-O-desosaminyl-11-(3-(4-hydroxyphenyl)-propyl)hydrazo-9-methoxyimino-6-O-methyl-3-oxoerythronolide A, 11,12-carbamate;

9-Deoxo-11-deoxy-5-O-desosaminyl-11-(3-(3-methoxyphenyl)-propyl)hydrazo-9-hydroxyimino-6-O-methyl-3-oxoerythronolide A, 11,12-carbamate;

9-Deoxo-11-deoxy-5-O-desosaminyl-11-(3-(3-methoxyphenyl)-propyl)hydrazo-9-methoxyimino-6-O-methyl-3-oxoerythronolide A, 11,12-carbamate;

9-Deoxo-11-deoxy-5-O-desosaminyl-11-(2-phenylethyl)hydrazo-9-hydroxyimino-4-O-methyl-3-oxoerythronolide A, 11,12-carbamate;

9-Deoxo-11-deoxy-5-O-desosaminyl-11-(2-phenylethyl)hydrazo-9-methoxyimino-6-O-methyl-3-oxoerythronolide A, 11,12-carbamate;

9-Deoxo-11-deoxy-5-O-desosaminyl-11-(4-phenylbutyl)hydrazo-9-hydroxyimino-6-O-methyl-3-oxoerythronolide A, 11,12-carbamate;

9-Deoxo-11-deoxy-5-O-desosaminyl-11-(4-phenyibutyl)hydrazo-9-methoxyimino-4-O-methyl-3-oxoerythronolide A, 11,12-carbamate;

9-Deoxo-11-deoxy-5-O-desosaminyl-11-(3-furan-2-yl-propyl)hydrazo-9-hydroxyimino-6-O-methyl-3-oxoerythronolide A, 11,12-carbamate;

9-Deoxo-11-deoxy-5-O-desosaminyl-11-(3-furan-2-yl-propyl)hydrazo-9-methoxyimino-6-O-methyl-3-oxoerythronolide A, 11,12-carbamate;

9-Deoxo-11-deoxy-5-O-desosaminyl-11-(3-thiophen-2-yl-propyl)hydrazo-9-hydroxyimino-6-O-methyl-3-oxoerythronolide A, 11,12-carbamate;

9-Deoxo-11-deoxy-5-O-desosaminyl-11-(3-thiophen-2-yl-propyl)hydrazo-9-methoxyimino-6-O methyl-3-oxoerythronolide A, 11,12-carbamate;

9-Deoxo-11-deoxy-5-O-desosaminyl-11-(3-pyrrol-1-yl-propyl)hydrazo-9-hydroxyimino-6-O-methyl-3-oxoerythronolide A, 11,12-carbamate;

9-Deoxo-11-deoxy-5-O-desosaminyl-11-(3-pyrrol-1-yl-propyl)hydrazo-9-methoxyimino-6-O-methyl-3-oxoerythronolide A, 11,12-carbamate;

9-Deoxo-11-deoxy-5-O-desosaminyl-11-(3-pyrazol-1-yl-propyl)hydrazo-9-hydroxyimino-6-O-methyl-3-oxoerythronoiide A, 11,12-carbamate;

9-Deoxo-11-deoxy-5-O-desosaminyl-11-(3-pyrazol-1-yl-propyl)hydrazo-9-methoxyimino-6-O-methyl-3-oxoerythronolide A, 11,12-carbamate;

9-Deoxo-11-deoxy-5-O-desosaminyl-11-(3-(2-pyridin-3-yl-thiazol-4-yl)-propyl)hydrazo-9-hydroxyimino-6-O-methyl-3-oxoerythronolide A, 11,12-carbamate;

9-Deoxo-11-deoxy-5-O-desosaminyl-11-(3-(2-pyridin-3-yl-thiazol-4-yl)-propyl)hydrazo-9-methoxyimino-6-O-methyl-3-oxoerythronolide A, 11,12-carbamate;

9-Deoxo-11-deoxy-5-O-desosaminyl-11-(3-(2-phenyl-thiazol-5-yl)-propyl)hydrazo-9-hydroxyimino-6-O-methyl-3-oxoerythronolide A, 11,12-carbamate;

9-Deoxo-11-deoxy-5-O-desosaminyl-11-(3-(2-phenyl-thiazol-5-yl)-propyl)hydrazo-9-methoxyimino-6-O-methyl-3-oxoerythronolide A, 11,12-carbamate;

9-Deoxo-11-deoxy-5-O-desosaminyl-11-(3-(4-phenyl-1H-imidazol-2-yl)-propyl)hydrazo-9-hydroxyimino-6-O-methyl-3-oxoerythronolide A, 11,12-carbamate;

9-Deoxo-11-deoxy-5-O-desosaminyl-11-(3(4-phenyl-1H-imidazol-2-yl)-propyl)hydrazo-9-methoxyimino-6-O-methyl-3-oxoerythronolide A, 11,12-carbamate;

9-Deoxo-11-deoxy-5-O-desosaminyl-10-epi-11-hydrazo-9-benzoxyimino-6-O-methyl-3-oxoerythronolide A, 11,12-carbamate;

9-Deoxo-11-deoxy-5-O-desosaminyl-10-epi-11-hydrazo-9-hydroxyimino-6-O-methyl-3-oxoerythronolide A, 11,12-carbamate; and the pharmaceutically acceptable salts of the foregoing compounds.

The invention also relates to a pharmaceutical composition for the treatment of a bacterial infection or a protozoa infection in a mammal, fish, or bird which comprises a therapeutically effective amount of a compound of formula I, or a pharmaceutically acceptable salt thereof, and a pharmaceutically acceptable carrier.

The invention also relates to a method of treating a bacterial infection or a protozoa infection in a mammal, fish, or bird which comprises administering to said mammal, fish or bird a therapeutically effective amount of a compound of formula I or a pharmaceutically acceptable salt thereof.

The term “treatment”, as used herein, unless otherwise indicated, includes the treatment or prevention of a bacterial infection or protozoa infection as provided in the method of the present invention.

As used herein, unless otherwise indicated, the term “bacterial infection(s)” or “protozoa infection” includes bacterial infections and protozoa infections that occur in mammals, fish and birds as well as disorders related to bacterial infections and protozoa infections that may be treated or prevented by administering antibiotics such as the compounds of the present invention. Such bacterial infections and protozoa infections and disorders related to such infections include the following: pneumonia, otitis media, sinusitus, bronchitis, tonsillitis, and mastoiditis related to infection by Streptococcus pneumoniae, Haemophilus influenzae, Moraxella catarrhais, Staphylococcus aureus, or Peptostreptococcus spp.; pharynigitis, rheumatic fever, and glomerulonephritis related to infection by Streptococcus pyogenes, Groups C and G streptococci, Clostridium diptheriae, or Actinobacillus haemolyticum; respiratory tract infections related to infection by Mycoplasma pneumoniae, Legionella pneumophila, Streptococcus pneumoniae, Haemophilus influenzae, or Chiamydia pneumoniae; uncomplicated skin and soft tissue infections, abscesses and osteomyelitis, and puerperal fever related to infection by Staphylococcus aureus, coagulase-positive staphylococci (i.e., S. epidermidis, S. hemolyticus, etc.), Streptococcus pyogenes, Streptococcus agalactiae, Streptococcal groups C-F (minute-colony streptococci), viridans streptococci, Corynebactenum minutissimum, Clostndium spp., or Bartonella henselae; uncomplicated acute urinary tract infections related to infection by Staphylococcus saprophyticus or Enterococcus spp.; urethritis and cervicitis; and sexually transmitted diseases related to infection by Chiamydia trachomatis, Haemophilus ducreyi, Treponema pallidum, Ureaplasma urealyticum, or Neisenia gonornheae; toxin diseases related to infection by S. aureus (food poisoning and Toxic shock syndrome), or Groups A, B, and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by Chlamydia trachomatis, Neisseria gononhoeae, S. aureus, S. pneumoniae, S. pyogenes, H. influenzae, or L Usteria spp.; disseminated Mycobacterium avium complex (MAC) disease related to infection by Mycobacterium avium, or Mycobacterium intracellulare; gastroenteritis related to infection by Campylobacter jejuni; intestinal protozoa related to infection by Cryptosporidium spp.; odontogenic infection related to infection by viridans streptococci; persistent cough related to infection by Bordetella pertussis; gas gangrene related to infection by Clostridium perfringens or Bacteroides spp.; and atherosclerosis related to infection by Helicobacter pyloi or Chiamydia pneumoniae. Bacterial infections and protozoa infections and disorders related to such infections that may be treated or prevented in animals include the following: bovine respiratory disease related to infection by P. haem., P. multocida, Mycoplasma bovis, or Bordetella spp.; cow enteric disease related to infection by E. coli or protozoa (i.e., coccidia, cryptosporidia, etc.); dairy cow mastitis related to infection by Staph. aureus, Strep. uberis, Strep. agalactiae, Strep. dysgalactiae, Klebsiefla spp., Corynebacterium, or Enterococcus spp.; swine respiratory disease related to infection by A. pleuro., P. multocida, or Mycoplasma spp.; swine enteric disease related to infection by E. coli, Lawsonia intracellularis, Salmonella, or Serpulina hyodyisinteriae; cow footrot related to infection by Fusobacterium spp.; cow metritis related to infection by E. coli; cow hairy warts related to infection by Fusobacterium necrophorum or Bacteroides nodosus; cow pink-eye related to infection by Moraxella bovis; cow premature abortion related to infection by protozoa (i.e. neosporium); urinary tract infection in dogs and cats related to infection by E. coil; skin and soft tissue infections in dogs and cats related to infection by Staph. epidermidis, Staph. internedius, coagulase neg. Staph. or P. multocida; and dental or mouth infections in dogs and cats related to infection by Alcaligenes spp., Bacteroides spp., Clostndium spp., Enterobacter spp., Eubactetium, Peptostreptococcus, Porphyromonas, or Prevotella. Other bacterial infections and protozoa infections and disorders related to such infections that may be treated or prevented in accord with the method of the present invention are referred to in J. P. Sanford et al., “The Sanford Guide To Antimicrobial Therapy,” 26th Edition, (Antimicrobial Therapy, Inc., 1996).

The invention also relates to a process of preparing the compound of formula I, and pharmaceutically acceptable salts thereof, wherein R¹, R², R⁶ and X are as defined above, which comprises treating a compound of the formula

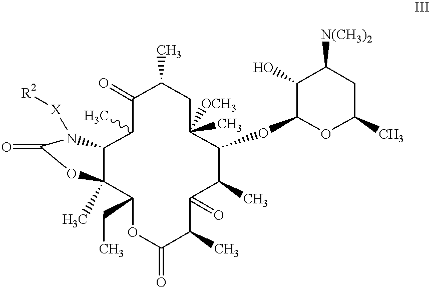

wherein X and R² are as defined above, with a compound of the formula R¹ONH₂.HCl or R¹ONH₂, wherein R¹ is as defined for said compound of formula I, in the presence of an acid, in a polar solvent such as methanol, ethanol, or isopropyl alcohol. Preferably, said acid is Py.HCl, wherein Py denotes pyridine, or Et₃N.HCl.

In the chemical structures depicted herein, a wavy line indicates that the stereochemistry at the chiral center to which the wavy line is connected is either an R or S configuration where the wavy line is connected to a carbon atom. In the compound of formula I, the wavy line at position 10 of the macrolide ring indicates that the methyl group can be either R or S configuration at that position. In the compound of formula I, the wavy line connected to the oxime nitrogen at position 9 of the macrolide ring indicates that the —OR¹ moiety is in an E or Z configuration.

The term “halo”, as used herein, unless otherwise indicated, means fluoro, chloro, bromo or iodo. Preferred halo groups are fluoro, chloro and bromo.

The term “alkyl”, as used herein, unless otherwise indicated, includes saturated monovalent hydrocarbon radicals having straight, cyclic or branched moieties, or mixdures thereof. Said alkyl group may include one or two double or triple bonds. It is understood that for cyclic moieties at least three carbon atoms are required in said alkyl group.

The term “alkanoyl”, as used herein, unless otherwise indicated, includes —C(O)-alkyl groups wherein “alkyl” is as defined above.

The term “aryl”, as used herein, unless otherwise indicated, includes an organic radical derived from an aromatic hydrocarbon by removal of one hydrogen, such as phenyl or naphthyl.

The term “5-10 membered heterocyclyl”, as used herein, unless otherwise indicated, includes aromatic and non-aromatic heterocyclic groups containing one or more heteroatoms each selected from O, S and N, wherein each heterocyclic group has from 5-10 atoms in its ring system. The heterocyclic groups include benzo-fused ring systems and ring systems substituted with one or more oxo moieties. An example of a 5 membered heterocyclic group is thiazolyl, and an example of a 10 membered heterocyclic group is quinolinyl. Examples of non-aromatic heterocyclic groups are pyrrolidinyl, piperidino, morpholino, thiomorpholino and piperazinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl and thiazolyl. Heterocyclic groups having a fused benzene ring include benzimidazolyl.

The phrase “pharmaceutically acceptable salt(s)”, as used herein, unless otherwise indicated, includes salts of acidic or basic groups which may be present in the compounds of formula I. The compounds of formula I that are basic in nature are capable of forming a wide variety of salts with various inorganic and organic acids. The acids that may be used to prepare pharmaceutically acceptable acid addition salts of such basic compounds of formula I are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, ptoluenesulfonate and pamoate [i.e., ,1,1′-methylene-bis-(2-hydroxy-3-naphthoate)] salts.

Those compounds of the formula I that are acidic in nature, are capable of forming base salts with various pharmacologically acceptable cations. Examples of such salts include the alkali metal or alkaline earth metal salts and particularly, the sodium and potassium salts.

The present invention also includes all radiolabelled forms of the compounds of formula I, and pharmaceutically acceptable salts thereof, wherein the radiolabel is selected from ³H, ¹¹C and ¹⁴C. Such radiolabelled compounds are useful as research or diagnostic tools.

Certain compounds of formula I may have asymmetric centers and therefore exist in different enantiomeric forms. This invention relates to the use of all optical isomers and stereoisomers of the compounds of formula I and mixtures thereof. In particular, the invention includes both the R and S configurations of the methyl group at C-10 of the macrolide ring of formula I, and both the E and Z configurations of the —OR¹ group connected to the nitrogen of the oxime moiety at C-9 of the macrolide ring of formula I. The compounds of formula I may also exist as tautomers. This invention relates to the use of all such tautomers and mixtures thereof.

### DETAILED DESCRIPTION OF THE INVENTION

The preparation of the compounds of the present invention is illustrated in the following Schemes.

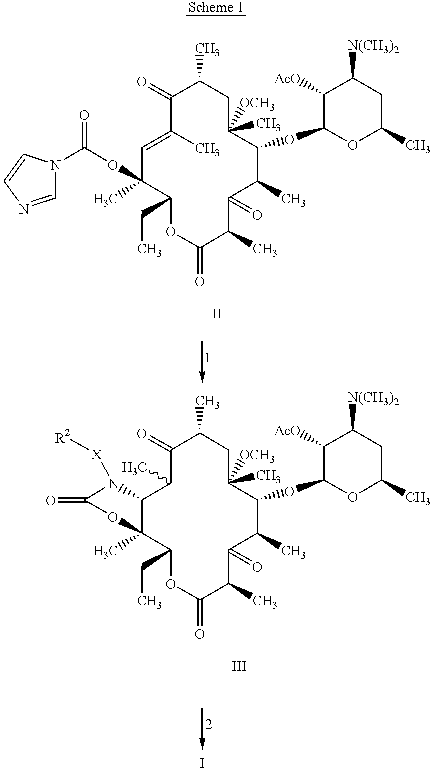

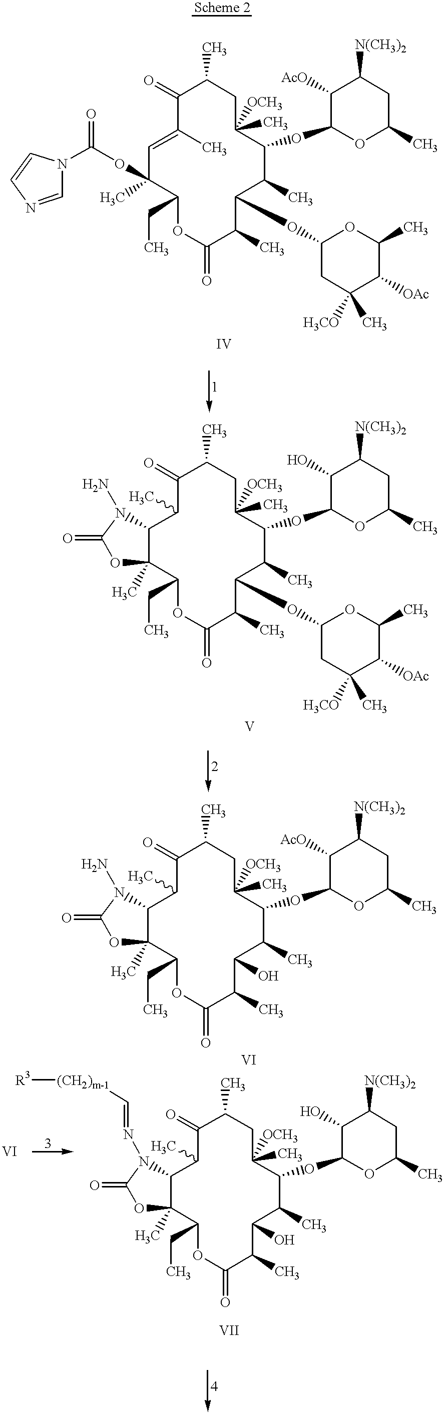

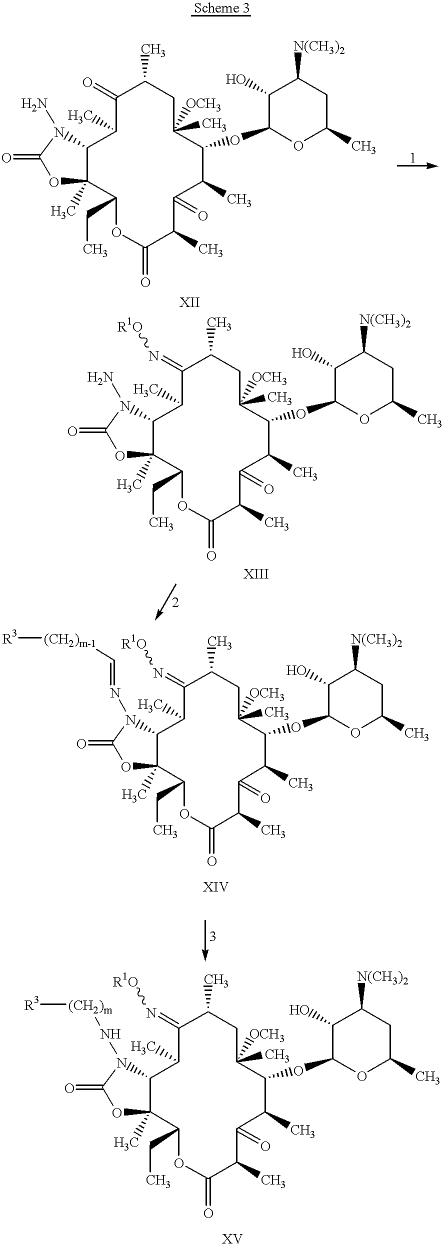

In the above Schemes, “Ac” indicates an acetyl group. The compounds of the present invention are readily prepared. Scheme 1 illustrates the general synthesis of the compounds of the present invention. In Scheme 1, the starting compound of formula II can be prepared as described in U.S. Pat. No. 5,543,400 (issued Aug. 6, 1996). In general, the intermediate compound of formula III can be prepared as described in U.S. Pat. No. 5,543,400, referred to above, U.S. Pat. No. 5,527,780 (issued Jun. 18, 1996), United Kingdom patent application number 2,288,174 (published Oct. 11, 1995), and G. Griesgraber et al., “3-Keto-11,12-carbazate Derivatives of 6-O-Methylerythromycin A,” Journal of Antibiotics, 49(5), 465-477 (1996).

In step 1 of Scheme 1, compounds of the formula ll, wherein X is —CR⁷R⁸— and R² is as defined above, can be prepared by treating a compound of the formula II with a compound of the formula H₂N—X—R², wherein X and R² are as indicated for said compound of formula III, in a solvent such as acetonitrile, dimethylformamide (DMF), tetrahydrofuran (THF), dimethoxy ethane or dimethylsulfoxide (DMSO), at a temperature within the range of about 50° C. to 90° C. for a period of about 4 to 10 hours. The preparation of compounds of the formula III wherein X is —CR⁷R⁸— is described in further detail in U.S. Pat. Nos. 5,543,400 and 5,527,780, referred to above. Compounds of the formula III, wherein X is —NH— and R² is as defined above, can be prepared by treating a compound of the formula II with a compound of the formula H₂NNHR², wherein R² is as defined above, in a solvent such as acetonitrile, dioxane, or DMSO, at a temperature within the range of about 40° C. to 90° C. for a period of about 12 hours. The preparation of the compound of formula III wherein X is —NH— is described in further detail in United Kingdom patent application number 2,288,174, referred to above. In step 2 of Scheme 1, compounds of the formula I can be prepared by treating a compound of the formula III with a compound of the formula R¹ONH₂.HCl or R¹ONH₂, wherein R¹ is as defined above, in the presence of an acid, such as Py.HCl, wherein Py denotes pyridine, or Et₃N.HCl, in a polar solvent, preferably methanol, ethanol, or isopropyl alcohol, at a temperature within the range of about 65° C. to 95° C. for a period of about 10 hours to 6 days.

Scheme 2 illustrates an alternative method of preparing compounds of the formula I wherein X is —NH—. In Scheme 2, the compound of formula IV can be prepared according to the procedures described in Baker et al., Journal of Organic Chemistry, 53, 2340 (1988). In step 1 of Scheme 2, the compound of formula V can be prepared by treating the compound of formula IV with hydrazine according to the procedure described above for the preparation of the compound of formula III wherein X is —NH—. Preferably, the compound of formula V is prepared by treating a compound of formula IV with anhydrous hydrazine in a solvent such as MeCN or DMF at a temperature of from about 60° C. to 90° C. for about 12 hours. In step 2 of Scheme 2, the compound of formula VI can be prepared by treating a compound of formula V with an acid, such as hydrochloric acid, in a solvent such as methanol or ethanol. In step 3 of Scheme 2, the compound of formula VII can be prepared by treating a compound of formula VI with a compound of the formula R³—(CH₂)ₘ₋₁—C(O)H, wherein m is 1 to 7 and R³ is as defined above, in an anhydrous solvent, such as anhydrous ethanol or isopropanol, at a temperature within the range of about 80° C. to 90° C.

In steps 4 and 5 of Scheme 2, the hydroxy group at position C-2′ of the compound of formula VII is protected as an acetate by treating the compound with acetic anhydride to form the compound of formula VIII, followed by oxidation of the hydroxy group at position 3 to provide a carbonyl group. Preferably, this is done by treating the compound of formula VII with acetic anhydride in a solvent such as CH₂Cl₂ at ambient temperature to provide the compound of formula VIII. The compound of formula IX can be prepared by treating a compound of formula VIII, in a solvent such as CH₂CI₂, with DMSO, 1-ethyl-3-(3-dimethylaminopropyl)carbodiimide (EDAC) and pyridinium trifluoroacetate (Py.TFA) at ambient temperature. The compound of formula X can be prepared by treating the compound of formula IX with a reducing agent such as NaBH₃CN in a solvent such as methanol at ambient temperature. The compound of formula XI can be prepared as described above for step 2 of Scheme 1.

Scheme 3 illustrates an additional method of preparing compounds of the formula I wherein X is —NH—. In Scheme 3, the compound of formula XII can be prepared according to the procedures described in Griesgraber et al., Journal of Antibiotics, 49(5), 465-477 (1966). In step 1 of Scheme 3, the compound of formula XII can be prepared by treating the compound of formula XII with a compound of the formula R¹ONH₂.HCl or R¹ONH₂ in the presence of an acid such as Py.HCl, wherein Py denotes pyridine, or Et₃N.HCl, in a polar solvent, preferably ethanol, methanol, or isopropyl alcohol, at a temperature within the range of about 65° C. to 95° C. for a period of about 10 hours to 4 days. In step 2 of Scheme 3, the compound of formula XIII can be converted to the compound of formula XIV according to the procedure of step 3 of Scheme 2. In step 3 of Scheme 3, the compound of formula XIV can be converted to the compound of formula XV according to the procedure of step 6 of Scheme 2.

The compounds of the present invention may have asymmetric carbon atoms. Such diasteromeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomric mixture by reaction with an appropriate optically active compound (e.g., alcohol), separating the diastereomers and converting (e.g., hydrolyzing) the individual diastereomers to the corresponding pure enantiomers. All such isomers, including diastereomer mixtures and pure enantiomers are considered as part of the invention.

The compounds of formula I that are basic in nature are capable of forming a wide variety of different salts with various inorganic and organic acids. Although such salts must be pharmaceutically acceptable for administration to animals, it is often desirable in practice to initially isolate the compound of formula I from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of the formula I that are acidic in nature, are capable of forming base salts with various pharmacologically acceptable cations. Examples of such salts include the alkali metal or alkaline-earth metal salts and particularly, the sodium and potassium salts. These salts may be prepared by conventional techniques. The chemical bases which are used as reagents to prepare the pharmaceutically acceptable base salts of this invention are those which form non-toxic base salts with the acidic compounds of formula I. Such non-toxic base salts include those derived from such pharmacologically acceptable cations as sodium, potassium calcium and magnesium, etc. These salts can be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

The activity of the compounds of the present invention against bacterial and protozoa pathogens is demonstrated by the compound's ability to inhibit growth of defined strains of human (Assay I) or animal (Assays II and III) pathogens.

### Assay I

Assay I, described below, employs conventional methodology and interpretation criteria and is designed to provide direction for chemical modifications that may lead to compounds that circumvent defined mechanisms of macrolide resistance. In Assay I, a panel of bacterial strains is assembled to include a variety of target pathogenic species, including representatives of macrolide resistance mechanisms that have been characterized. Use of this panel enables the chemical structure/activity relationship to be determined with respect to potency, spectrum of activity, and structural elements or modifications that may be necessary to obviate resistance mechanisms. Bacterial pathogens that comprise the screening panel are shown in the table below. In many cases, both the macrolide-susceptible parent strain and the macrolide-resistant strain derived from it are available to provide a more accurate assessment of the compound's ability to circumvent the resistance mechanism. Strains that contain the gene with the designation of ermA/ermB/ermC are resistant to macrolides, lincosamides, and streptogramin B antibiotics due to modifications (methylation) of 23S rRNA molecules by an Erm methylase, thereby generally prevent the binding of all three structural classes. Two types of macrolide efflux have been described; msrA encodes a component of an efflux system in staphylococci that prevents the entry of macrolides and streptogramins while mefA/E encodes a transmembrane protein that appears to efflux only macrolides. Inactivation of macrolide antibiotics can occur and can be mediated by either a phosphorylation of the 2′-hydroxyl (mph) or by cleavage of the macrocyclic lactone (esterase). The strains may be characterized using conventional polymerase chain reaction (PCR) technology and/or by sequencing the resistance determinant. The use of PCR technology in this application is described in J. Sutcliffe et al., “Detection Of Erythromycin-Resistant Determinants By PCR”, Antimicrobial Agents and Chemotherapy, 40(11), 2562-2566 (1996). The assay is performed in microtiter trays and interpreted according to Performance Standards for Antimicrobial Disk Susceptibility Tests—Sixth Edition; Approved Standard, published by The National Committee for Clinical Laboratory Standards (NCCLS) guidelines; the minimum inhibitory concentration (MIC) is used to compare strains. Compounds are initially dissolved in dimethylsulfoxide (DMSO) as 40 mg/ml stock solutions.

| Strain Designation | Macrolide Resistance Mechanism(s) |
|---|---|
| Staphylococcus aureus 1116 | susceptible parent |
| Staphylococcus aureus 1117 | ermB |
| Staphylococcus aureus 0052 | susceptible parent |
| Staphylococcus aureus 1120 | ermC |
| Staphylococcus aureus 1032 | msrA, mph, esterase |
| Staphylococcus hemolyticus 1006 | msrA, mph |
| Streptococcus pyogenes 0203 | susceptible parent |
| Streptococcus pyogenes 1079 | ermB |
| Streptococcus pyogenes 1062 | susceptible parent |
| Streptococcus pyogenes 1061 | ermB |
| Streptococcus pyogenes 1064 | ermB |
| Streptococcus agalactiae 1024 | susceptible parent |
| Streptococcus agalactiae 1023 | ermB |
| Streptococcus pneumoniae 1016 | susceptible |
| Streptococcus pneumoniae 1046 | ermB |
| Streptococcus pneumoniae 1095 | ermB |
| Streptococcus pneumoniae 1175 | mefE |
| Streptococcus pneumoniae 0085 | susceptible |
| Haemophilus influenzae 0131 | susceptible |
| Moraxella catarrhalis 0040 | susceptible |
| Moraxella catarrhalis 1055 | erythromycin intermediate |
| | resistance |
| Escherichia coli 0266 | susceptible |

Assay II is utilized to test for activity against Pasteurella multocida and Assay III is utilized to test for activity against Pasteurella haemolytica.

### Assay II

This assay is based on the liquid dilution method in microliter format. A single colony of P. multocida (strain 59A067) is inoculated into 5 ml of brain heart infusion (BHI) broth. The test compounds are prepared by solubilizing 1 mg of the compound in 125 μl of dimethylsulfoxide (DMSO). Dilutions of the test compound are prepared using uninoculated BHI broth. The concentrations of the test compound used range from 200 μg/ml to 0.098 μg/ml by two-fold serial dilutions. The P. multocida inoculated BHI is diluted with uninoculated BHI broth to make a 10⁴ cell suspension per 200 μl. The BHI cell suspensions are mixed with respective serial dilutions of the test compound, and incubated at 37° C. for 18 hours. The minimum inhibitory concentration (MIC) is equal to the concentration of the compound exhibiting 100% inhibition of growth of P. multocida as determined by comparison with an uninoculated control.

### Assay ll

This assay is based on the agar dilution method using a Steers Replicator. Two to five colonies isolated from an agar plate are inoculated into BHI broth and incubated overnight at 37° C. with shaking (200 rpm). The next morning, 300 μl of the fully grown P. haemolytica preculture is inoculated into 3 ml of fresh BHI broth and is incubated at 37° C. with shaking (200 rpm). The appropriate amounts of the test compounds are dissolved in ethanol and a series of two-fold serial dilutions are prepared. Two ml of the respective serial dilution is mixed with 18 ml of molten BHI agar and solidified. When the inoculated P. haemolytica culture reaches 0.5 McFarland standard density, about 5 μl of the P. haemolytica culture is inoculated onto BHI agar plates containing the various concentrations of the test compound using a Steers Replicator and incubated for 18 hours at 37° C. Initial concentrations of the test compound range from 100-200 μg/ml. The MIC is equal to the concentration of the test compound exhibiting 100% inhibition of growth of P. haemolytica as determined by comparison with an uninoculated control.

The in vivo activity of the compounds of formula (I) can be determined by conventional animal protection studies well known to those skilled in the art, usually carried out in mice.

Mice are allotted to cages (10 per cage) upon their arrival, and allowed to acclimate for a minimum of 48 hours before being used. Animals are inoculated with 0.5 ml of a 3×10³ CFU/ml bacterial suspension (P. multocida strain 59A006) intraperitoneally. Each experiment has at least 3 non-medicated control groups including one infected with 0.1× challenge dose and two infected with 1× challenge dose; a 10× challenge data group may also be used. Generally, all mice in a given study can be challenged within 30-90 minutes, especially if a repeating syringe (such as a Cornwall® syringe) is used to administer the challenge. Thirty minutes after challenging has begun, the first compound treatment is given. It may be necessary for a second person to begin compound dosing if all of the animals have not been challenged at the end of 30 minutes. The routes of administration are subcutaneous or oral doses. Subcutaneous doses are administered into the loose skin in the back of the neck whereas oral doses are given by means of a feeding needle. In both cases, a volume of 0.2 ml is used per mouse. Compounds are administered 30 minutes, 4 hours, and 24 hours after challenge. A control compound of known efficacy administered by the same route is included in each test. Animals are observed daily, and the number of survivors in each group is recorded. The P. multocida model monitoring continues for 96 hours (four days) post challenge.

The PD₅₀ is a calculated dose at which the compound tested protects 50% of a group of mice from mortality due to the bacterial infection which would be lethal in the absence of drug treatment.

The compounds of formula I, and the pharmaceutically acceptable salts thereof (hereinafter “the active compounds”), may be adminstered through oral, parenteral, topical, or rectal routes in the treatment or prevention of bacterial or protozoa infections. In general, these compounds are most desirably administered in dosages ranging from about 0.2 mg per kg body weight per day (mg/kg/day) to about 200 mg/kg/day in single or divided doses (i.e., from 1 to 4 doses per day), although variations will necessarily occur depending upon the species, weight and condition of the subject being treated and the particular route of administration chosen. However, a dosage level that is in the range of about 4 mg/kg/day to about 50 mglkg/day is most desirably employed. Variations may nevertheless occur depending upon the species of mammal, fish or bird being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effects, provided that such larger doses are first divided into several small doses for administration throughout the day.

The active compounds may be administered alone or in combination with pharmaceutically acceptable carriers or diluents by the routes previously indicated, and such administration may be carried out in single or multiple doses. More particularly, the active compounds may be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the active compounds are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral adinistration, the active compound may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of an active compound in either sesame or peanut oil or in aqueous propylene glycol may be employed. The aqueous solutions should be suitably buffered (preferably pH greater than 8) if necessary and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques will known to those skilled in the art.

Additionally, it is also possible to administer the active compounds of the present invention topically and this may be done by way of creams, jellies, gels, pastes, patches, ointments and the like, in accordance with standard pharmaceutical practice.

For administration to animals other than humans, such as cattle or domestic animals, the active compounds may be administered in the feed of the animals or orally as a drench composition.

The active compounds may also be adminstered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The active compounds may also be coupled with soluble polymers as targetable drug carriers. Such polymers can include polyvinylpyrrolidone, pyran copolymer, polyhydroxypropylmethacrylamide phenyl, polyhydroxyethylaspartamide-phenol, or polyethyleneoxide-polylysine substituted with palmitoylresidues. Furthermore, the active compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross-linked or amphipathic block copolymers of hydrogels.

The Examples provided below illustrate specific embodiments of the invention, but the invention is not limited in scope to the Examples specifically exemplified.

### EXAMPLE 1

### 4″-Acetyl-11-deoxy-11-hydrazo-6-O-methylerythromycin A, 11,12-carbamate

To a solution of 10,11-anhydro-2′,4′-di-O-acetyl-12-O-imidazolylcarbonyl-6-O-methylerythromycin A (460 mg, 0.51 mmol) (which was prepared following the procedures of Baker et al., J. Org. Chem., 1988, 53, 2340), in MeCN at room temperature was added anhydrous NH₂NH₂ (0.16 mL, 5.1 mmol) and the resulting solution was heated at 60° C. for 12 hours. MeCN was removed in vacuo, brine and EtOAc were added, and the aqueous layer was extracted with EtOAc (3 times). The combined organic layers were washed with brine, dried over anhydrous MgSO₄, and concentrated in vacuo. The crude product was purified by silica gel flash chromatography (1.5% Et₃N-1.5% MeOH-97% MeOBu-f) to afford the title compound as a white solid (261 mg, 62%) and 4″-acetyl-10-epi-11-deoxy-11-hydrazo-6-O-methylerythromycin A, 11,12-carbamate as a white solid (38 mg, 10%).

For the title compound: ¹H NMR (400 MHz, CDCl₃) δ: 5.02 (1H, dd, J=1.6, 10.8 Hz), 4.94 (1H, d, J=4.8 Hz), 4.65 (1H, d, J=10.0 Hz), 4.53 (1H, d, J=7.2 Hz), 4.33 (1H, m), 3.82-3.30 (m), 329 (3H, s), 3.20-3.00 (m), 3.00 (3H, s), 2.92-2.31 (m), 2.27 (6H, s), 2.08 (3H, s), 2.0-1.4 (m), and 0.82 (3H, t, J=7.6 Hz); MS: m/z 830 (M+H).

For 4″-acetyl-10-epi-11-deoxy-11-hydrazo-6-O-methylerythromycin A, 11,12-Carbamate: δ: ¹H NMR (400 MHz, CDCl₃): 5.05 (1H, d, J=4.4 Hz), 4.87 (1H, dd, J=1.6, 10.8 Hz), 4.64 (1H, d, J=10.0 Hz), 4.43 (1H, d, J=7.6 Hz), 4.33 (1H, m), 3.84-3.32 (m), 3.30 (3H, s), 3.18 (3H, s), 3.2-2.3 (m), 2.23 (6H, s), 2.08 (3H, s), 1.62 (3H, s), 1.30 (3H, s), 0.97 (3H, d, J=6.8 Hz), and 0.84 (3H, t, J=7.2 Hz); MS: m/z 830 (M+H).

### EXAMPLE 2

### 11-Deoxy-5-O-desosaminyl-11-hydrazo-6-O-methylerythronolide A, 11,12-carbamate

A solution of 4-acetyl-11-deoxy-11-hydrazo-6-O-methylerythromycin A, 11,12-carbamate (40 mg, 0.048 mmol) in EtOH-2N HCl (1:2) was stirred at room temperature overnight and the reaction mixture was poured into a cold solution of saturated NaHCO₃. The aqueous layer was extracted with CHCl₃ (3 times). The combined organic layers were washed with brine, dried over anhydrous MgSO₄, and concentrated in vacuo. The crude product was purified by silica gel flash chromatography (5% MeOH-0.5% NH₃.H₂O-94.5% CH₂CI₂) to afford the title compound as a white solid (26.1 mg, 86%):

¹H NMR (400 MHz, CDCl₃) δ: 5.09 (1H, dd, J=2.4, 10.8 Hz), 4.45 (1H, s), 4.38 (1H, d, J=7.6 Hz), 3.9-3.0 (m), 2.95 (3H, s), 2.75-2.45 (m), 2.27 (6H, s), 2.0-1.4 (m), 1.40 (3H, s), 1.34 (3H, s), 1.24 (3H, d, J=6.8 Hz), 1.23 (3h, d, J=6.0 Hz), 1.12 (3H, d, J=7.2 Hz), 1.09 (3H, d, J=7.2 Hz), 1.07 (3H, d, J=6.8 Hz), and 0.80 (3H, t, J=7.6 Hz).

¹³C NMR (100.6 MHz, CDCl₃) δ: 216.90, 175.69, 156.45, 106.65, 88.29, 81.46, 78.74, 78.36, 75.78, 70.63, 70.23, 65.71, 63.37, 49.30, 45.35, 44.66, 40.27 (2C), 39.57, 38.87, 36.07, 28.28, 22.01, 21.23, 18.88, 18.16, 15.19, 14.15, 13.74, 10.14, 8.22.

MS: m/z 629 (M+H).

### EXAMPLE 3

### 11-Deoxy-5-O-desosaminyl-11-(3-quinolin-4-yl-propylidene)hydrazo-6-O-methylerythronolide A, 11,12-carbamate

To a solution of 11-deoxy-5-O-desosaminyl-11-hydrazo-6-O-methyl-erythronolide A, 11,12-carbamate (1.35 g, 21.7 mmol) in anhydrous EtOH (20 mL) was added 3-(4-quinolinyl)propionaldehyde (0.57 g, 3.08 mmol) and the resulting solution was heated at 86° C. for two days. EtOH was evaporated in vacuo to give the title compound as a white solid (1.84 g, 97%).

¹H NMR (400 MHz, CDCl₃) δ: 8.81 (1H, d, J=4.4 Hz), 8.09 (1H, d, J=5.2 Hz), 8.07 (1H, d, J=5.2 Hz), 7.69 (1H, t, J=8.4 Hz), 7.56 (1H, t, J=7.2 Hz), 7.29 (1H, d, J=4.0 Hz), 5.08 (1H, dd, J=2.0, 10.8Hz), 4.53 (1H, s), 4.41 (1H, d, J=7.6 Hz), 4.2-3.2 (m), 2.99 (3H, s), 2.9-2.4 (m), 2.25 (6H, s), 2.0-1.5 (m), 1.48 (3H, s), 1.29 (3H, s), 1.30 (3H, d, J=6.4 Hz), 1.24 (3H, d, J=6.0 Hz), 1.15 (3H, d, J=6.4 Hz), 1.10 (3H, d, J=7.2 Hz), 1.07 (3H, d, J=6.4 Hz), and 0.87 (3H, t, J=7.2 Hz).

MS: m/z 794 (M+H).

### EXAMPLE 4

### 2′-Acetyl-11-deoxy-5-O-desosaminyl-11-(3-quinolin-4-yl-propylidene)hydrazo-6-O-methylerythronolide A, 11,12-carbamate

To a solution of 11-deoxy-5-O-desosaminyl-11-(3-quinolin-4-yl-propylidene)hydrazo-6-O-methylerythronolide A, 11,12-carbamate (2.07 g, 2.60 mmol) in CH₂Cl₂ (10 mL) was added acetic anhydride (0.49 mL, 5.20 mmol) and the resulting solution was stirred at room temperature for 5 hours. Saturated NaHCO₃ was added, the aqueous layer was extracted with CH₂Cl₂, and the combined organic layers were washed with brine, dried over anhydrous MgSO₄, and evaporated in vacuo to give the title compound as a white solid (1.79 g, 82%).

¹H NMR (400 MHz, CDCl₃) δ: 8.80 (1H, d, J=4:4 Hz), 8.08 (1H, d, J=8.4 Hz), 8.05 (1H, d, J=9.2 Hz), 8.01 (1H, t, J=4.8 Hz), 7.68 (1H, dt, J=1.2, 6.8 Hz), 7.55 (1H, dt, J=1.2, 6.8 Hz), 7.27 (1H, d, J=4.4 Hz), 5.02 (1H, dd, J=2.8, 10 Hz), 4.718 (1H, t, J=6.7 Hz), 4.40 (1H, s), 4.35 (1H, d, J=7.6 Hz), 4.19 (1H, d, J=8.0 Hz), 3.81 (1H, q, J=6.8 Hz), 3.6-2.4 (m), 2.71 (3H, s), 2.24 (6H, s), 1.9-1.5 (m), 1.55 (3H, s), 1.35 (3H, d, J=6.8 Hz), 1.33 (3H s), 1.22 (3H, d, J=6.0 Hz), 1.14 (3H, d, J=7.6 Hz), 1.10 (3H, d, J=6.8 Hz), 1.06 (3H, d, J=6.4 Hz), and 0.87 (3H, t, J=7.2 Hz).

MS: m/z 837 (M+H).

### EXAMPLE 5

### 2′-Acetyl-11-deoxy-5-O-desosaminyl-11-(3-quinolin-4-yl-propylidene)hydrazo-6-O-methyl-3-oxoerythronolide A, 11,12-carbamate

To a solution of 2′-acetyl-11-deoxy-5-O-desosaminyl-11-(3-quinolin-4-yl-propylidene)hydrazo-6-O-methylerythronolide A, 11,12-carbamate (1.69 g, 2.02 mmol) in CH₂Cl₂ (16 mL) was added DMSO (1.85 mL, 8.06 mmol, 10 eq), 1-ethyl-3-(3-dimethylaminopropyl)carbodiimide (EDAC) (1.55 g, 8.06 mmol, 3.1 eq), and Py.TFA (1.56 g, 8.06 mmol, 3.1 eq) and the resulting suspension was stirred at room temperature for 2 hours. Saturated NaHCO₃ was added, the aqueous layer was extracted with CH₂Cl₂ (3 times). The combined organic layers were washed with brine, dried over anhydrous MgSO₄, and concentrated in vacuo. The crude product was purified by silica gel flash chromatography (5% MeOH-0.5% NH₃.H₂O-94.5% CH₂Cl₂) to afford the title compound as a white solid (1.59 g, 94%).

¹H NMR (400 MHz, CDCl₃): 8.79 (1H, d, J=3.6 Hz), 8.08 (1H, d, J=8.8 Hz), 8.05 (1H, d, J=8.4 Hz), 8.00 (1H, t, J=4.8 Hz), 7.69 (1H, t, J=8.4 Hz), 7.55 (1H, t, J=7.2 Hz), 7.27 (1H, d, J=4.4Hz), 5.03 (1H, dd, J=2.4, 10.0 Hz), 4.70 (1H, dd, J=7.6, 10.4 Hz), 4.39 (1H, s), 4.34 (1H, d, J=7.6 Hz), 4.18 (1H, d, J=8.0 Hz), 3.82 (1H, q, J=6.8 Hz), 3.6-1.6 (m), 2.71 (3H, s), 2.22 (6H, s), 2.03 (3H, s), 1.54 (3H, s), 1.33 (3H, d, J=6.4 Hz), 1.28 (3H, s), 1.21 (3H, d, J=6.4 Hz), 1.14 (3H, d, J=7.6 Hz), 1.09 (3H, d, J=6.8 Hz), 1.06 (3H, d, J=6.8 Hz), 0.87 (3H, t, J=7.2 Hz).

MS: m/z 836 (M+H).

### EXAMPLE 6

### 11-Deoxy-5-O-desosaminyl-11-(3-quinolin-4-yl-propyl))hydrazo-6-O-methyl-3-oxoerythronolide A, 11,12-carbamate:

To a solution of 2′-acetyl-11-deoxy-5-O-desosaminyl-11-(3-quinolin-4-yl-propylidene)hydrazo-6-O-methyl-3-oxoerythronolide A, 11,12-carbamate (1.59 g, 1.90 mmol) in MeOH (25 mL) at room temperature was added NaBH₃CN (359 mg, 5.70 mmol, 3 eq) followed by HOAc (0.65 mL, 11.4 mmol, 6 eq) and the resulting reaction mixture was stirred at room temperature overnight. MeOH was removed in vacuo and the residue was dissolved in CH₂Cl₂. Saturated NaHCO₃ was added, the aqueous layer was extracted with CH₂Cl₂ (3 times), the combined organic layers were washed with brine, dried over anhydrous MGSOA, and concentrated in vacuo. The residue was dissolved in MeOH (50 mL) and the solution was heated under reflux for 1 hour. MeOH was then removed in vacuo and the crude product was purified by silica gel flash chromatography (5% MeOH-0.5% NH₃.H₂O-94.5% CH₂Cl₂) to afford the tile compound as a white solid (1.13 g, 75%).

¹H NMR (400 MHz, CDCl₃) δ: 8.75 (1H, d, J=4.27 Hz), 8.07 (2H, t, J=9.4 Hz), 7.64 (1H. dt, J=1.6, 6.8 Hz), 7.50 (1H, dt, J=1.6, 7.2 Hz), 7.26 (1H, d, J=4.4 Hz), 5.47 (1H, t, J=3.6 Hz), 4.98 (1H, d, J=10.8 Hz), 4.26 (1H, s), 4.24 (1H, s), 3.84 (1H, q, J=6.8 Hz), 3.71 (1H, s), 3.8-2.7 (m), 2.62 (3H, s), 2.S2.3 (m), 2.22 (6H, s), 2.2-1.5 (m), 1.44 (3H, s), 1.33 (3H, d, J=6.8 Hz), 1.31 (3H, s), 1.29 (3H, d , J=7.6 Hz), 1.21 (3H, d, J=6.0 Hz), 1.16 (3H, d, J 6.0 Hz), 1.04 (3H, d, J=6.8 Hz), and 0.78 (3H, t, J=7.2 Hz).

¹³C NMR (100.6 MHz, CDCl₃) δ (attached H's): 217.82 (0), 203.63 (0), 169.72 (0), 156.10 (0), 150.18 (1), 148.20 (2C, 0), 130.05 (1), 128.85 (1), 127.58 (0), 126.19 (1), 123.81 (1), 120.97 (1), 103.96 (1), 80.69 (0), 79.37 (1), 78.12 (0), 77.35 (1), 70.33 (1), 69.63 (1), 65.84 (1), 58.14 (1), 51.04 (1), 50.13 (3), 48.41 (2), 47.31 (1), 44.55 (1), 40.19 (2C, 3), 39.60 (2C, 2 and 1), 29.49 (2), 28.47 (2), 28.14 (2), 22.03 (2), 21.14 (3), 19.82 (3), 18.50 (3), 15.31 (3), 14.55 (3), 14.38 (3),14.21 (3), and 10.35 (3).

MS: m/z 797 (M+H).

### EXAMPLE 7

### 9-Deoxo-11-deoxy-5-O-desosaminyl-11-(3-quinolin-4-yl-propyl)hydrazo-9-hydroxyimino-6-O-methyl-3-oxoerythronolide A, 11,12-carbamate

Method I:

To a solution of 11-deoxy-5-O-desosaminyl-11-(3-quinolinyl-propyl)hydrazo-6-O-methyl-3-oxoerythronolide A, 11,12-carbamate (654 mg, 0.82 mmol) in i-PrOH (8.0 mL) was added NH₂OH.HCl (855 mg, 12.3 mmol, 15 eq) and the reaction mixture was heated at 90° C. for six days. i-PrOH was removed in vacuo and the residue was dissolved in CH₂Cl₂. Saturated NaHCO₃ was added, the aqueous layer was extracted with CH₂Cl₂ (3 times), the combined organic layers were washed with brine, dried over anhydrous MgSO₄, and concentrated in vacuo. The crude product was purified by preparative TLC (10% MeOH-1% NH₃.H₂O-89% CH₂Cl₂) to afford the title compound as a white solid (224 mg, 34%) and the recovered starting material (107 mg, 16%).

¹H NMR (400 MHz, CDCl₃) δ: 10.85 (1H, br s), 8.39 (1H, d, J=4.4 Hz), 8.00 (1H. d, J=8.4 Hz), 7.90 (1H, d, J=8.0 Hz), 7.60 (1H, t, J=7.2 Hz), 7.42 (1H, t, J=7.6 Hz), 6.82 (1H, d, J=4.8 Hz), 5.05 (1H, dd, J=2.0, 10.8 Hz), 4.30 (1H, dd, J=5.6, 7.2 Hz), 3.89 (1H, q, J=6.4 Hz), 3.92 (1H, s), 3.55 (1H, m), 3.27 (1H, m), 3.15-2.50 (m), 2.83 (3H, s), 2.35 (6H, s), 1.95 (1H, m), 1.8-1.2 (m), 1.55 (3H, s), 1.49 (3H, s), 1.35 (3H, d, J=6.8 Hz), 1.29 (3H, d, J=7.6 Hz), 1.26 (3H, d, J=6.0 Hz), 1.15 (3H, d, J=6.8 Hz), 1.03 (3H, d, J=6.8 Hz), and 0.825 (3H, t, J=7.2 Hz).

¹³C NMR (100.6 MHz, CDCl₃) δ (attached H's): 203.73 (0), 169.85 (0), 166.52 (0), 156.35 (0), 149.22 (1), 147.06 (0), 129.35 (1), 128.85 (1), 127.36 (0), 126.41 (1), 123.77 (1), 120.24 (1), 103.91 (1), 81.28 (0), 79.77 (1), 78.66 (0), 77.37 (1), 70.34 (1), 69.40 (1), 65.95 (1), 59.68 (1), 51.16 (1), 50.52 (3), 48.08 (2), 47.52 (1), 40.26 (2C, 3), 38.26 (2), 33.66 (1), 29.40 (2), 28.51 (2), 27.93 (2), 25.59 (1), 22.17 (2), 21.18 (3), 20.09 (3), 19.15 (3), 17.40 (3), 15.37 (3), 14.52 (3),14.36 (3), and 10.44 (3).

MS: m/z 812 (M+H).

Method II:

To a solution of 9-deoxo-11-deoxy-5-O-desosaminyl-11-(3-quinoiin4-yl-propylidene)hydrazo-9-hydroxyimino-6-O-methyl-3oxoerythronolide A, 11,12-carbamate (186 mg, 0.23 mmol), prepared according to the procedures as described in Example 17, in methanol was added HOAC (212 uL, 3.7 mmol) and NaBH₃CN (158 mg, 2.3 mmol) and the resulting mixture was stirred at room temperature for 12 h. MeOH was removed in vacuo and the residue was dissolved in CH₂Cl₂. Saturated NaHCO₃ was added, the aqueous layer was extracted with CH₂Cl₂ (3 times), the combined organic layers were washed with brine, dried over anhydrous MgSO₄, and concentrated in vacuo. The crude product was purified by silica gel flash chromatography (5% MeOH-0.5% NH₃-H₂O-94.5% CH₂Cl₂) to afford the title compound as a white solid (114 mg, 61%).

### EXAMPLE 8

### 9-Deoxo-11-deoxy-5-O-desosaminyl-11-(3-quinolin-4-yl-propyl)hydrazo-9-methoxyimino-6-O-methyl-3-oxoerythronolide A, 11,12-carbamate

Method I:

To a solution of 11-deoxy-5-O-desosaminyl-11-(3-quinolin-4-yl-propyl)hydrazo-6-O-methyl-3-oxoerythronolide A, 11,12-carbamate (50 mg, 0.063 mmol) in i-PrOH (1.0 mL) was added NH₂OMe.HCl (26 mg, 0.31 mmol, 5 eq) and the reaction mixture was heated at 90° C. for three days. i-PrOH was removed in vacuo and the residue was dissolved in CH₂Cl₂. Saturated NaHCO₃ was added, the aqueous layer was extracted with CH₂Cl₂ (3 times), the combined organic layers were washed with brine, dried over anhydrous MgSO₄, and concentrated in vacuo. The crude product was purified by preparative TLC (10% MeOH-1% NH₃.H₂O-89% CH₂Cl₂) to afford the title compound as a white solid (27 mg, 52%) and some recovered starting material.

¹H NMR (400 MHz, CDCl₃) δ: 8.75 (1H, d, J=4.4 Hz), 8.12 (1H, d, J=8.4 Hz), 8.06 (1H, d, J=8.8 Hz), 7.65 (1H, t, J=6.8 Hz), 7.50 (1H, t, J=6.8 Hz), 727 (1H, d, J=4.4 Hz), 6.04 (1H, br s), 5.01 (1H, d, J=9.6 Hz), 4.3-1.4 (m), 3.86 (1H, q, J=6.8 Hz), 3.70 (3H, s), 2.66 (3H, s), 2.31 (6H, s), 1.45 (3H, s), 1.37 (3H, s), 1.33 (3H, d, J=6.8 Hz), 1.27 (3H, d, J=7.2 Hz), 1.24 (3H, d, J=6.0 Hz), 1.10 (3H, d, J=6.4Hz), 0.98 (3H, d, J=6.8 Hz), and 0.77 (3H, t, J=7.6 Hz).

MS: m/z 826 (M+H).

Method II

The title compound was prepared from 9-deoxo-11-deoxy-5-O-desosaminyl-11-hydrazo-9-methoxyimino-6-0rmethyl-3-oxoerythronolide A, 11,12-carbamate and 3-quinolin-4-yl-propioaldehyde following the procedures as described in Example 11.

### EXAMPLE 9

### 9-Deoxo-11 deoxy-5-O-desosaminyl-11-(3-quinolin-4-yl-propyl))hydrazo-9-benzoxyimino-6-O-methyl-3-oxoerythronolide A, 11,12-carbamate

To a solution of 11-deoxy-5-O-desosaminyl-11-(3-quinolin-4-yl-propyl)hydrazo-6-O-methyl-3-oxoerythronolide A, 11,12-carbamate (40 mg, 0.050 mmol) in i-PrOH (1.0 mL) was added NH₂OBn.HCl (32 mg, 0.20 mmol) and the reaction mixture was heated at 90° C. for three days. i-PrOH was removed in vacuo and the residue was dissolved in CH₂Cl₂. Saturated NaHCO₃ was added, the aqueous layer was extracted with CH₂Cl₂ (3 times), the combined organic layers were washed with brine, dried over anhydrous MgSO₄, and concentrated in vacuo. The crude product was purified by preparative TLC (10% MeOH-1% NH₃.H₂O-89% CH₂Cl₂) to afford the title compound as a white solid (10 mg, 22%) and some recovered starting material.

¹H NMR (400 MHz, CDCl₃): 8.75 (1H, d, J=4.4 Hz), 8.10 (1H, d, J=8.4 Hz), 8.07 (1H, d, J=8.4 Hz), 7.65 (1H, t, J=7.2Hz), 7.49 (1H, t, J=8.4 Hz), 7.30 (6H, m), 5.82 (1H, brs), 4.99 (1H, .d, J=8.0 Hz), 4.95 (1H, d, J=12.4 Hz, AB), 4.85 (1-H, d, J=12.4 Hz, AB), 4.3-1.4 (m), 3.86 (1H, q, J=6.8 Hz), 2.67 (3H, s), 2.30 (6H, s), 1.44 (3H, s), 1.40 (3H, s), 1.33 (3H, d, J=6.8 Hz), 1.28 (3H, d, J=7.6 Hz), 1.25 (3H, d, J=6.0 Hz), 1.07 (3H, d, J=7.2 Hz), 0.95 (3H, d, J=6.8 Hz), and 0.75 (3H, t, J=7.2 Hz).

MS: m/z 902 (M+H).

### EXAMPLE 10

### 9-Deoxo-11-deoxy-5-O-desosaminyl-11-hydrazo-9-methoxyimino-6-O-methyl-3-oxoerythronolide A, 11,12-carbamate

To a solution of 11-deoxy-5-O-desosaminyl-11-hydrazo-6-O-methyl-3-oxoerythronolide A, 11,12-carbamate (600 mg, 0.96 mmol) in EtOH (9.0 mL) was added NH₂OMe.HCl (319 mg, 3.83 mmol, 4.0 eq) and the reaction mixture was heated at 80° C. for 48 hours. EtOH was removed in vacuo and the residue was dissolved in CH₂Cl₂. Saturated NaHCO₃ was added, the aqueous layer was extracted with CH₂Cl₂ (3 times), the combined organic layers were washed with brine, dried over anhydrous MgSO₄, and concentrated in vacuo to give the title compound as a white solid (602 mg).

¹H NMR (400 MHz, CDCl₃) δ: 3.77 (3H, s), 2.68 (3H, s), 2.25 (6H, s), 1.43 (s, 3H), 1.38 (3H, s), 1.33 (3H, d, J=7.2 Hz), 1.27 (3H, d, J=7.6 Hz), 1.23 (3H, d, J=6.0 Hz), 1.13 (3H, d, J=6.8 Hz), 0.98 (3H, d, J=6.8 Hz), and 0.83 (3H, t, J=7.6 Hz).

MS: m/z 657 (M+H).

### EXAMPLE 11

### 9-Deoxo-11-deoxy-5-O-desosaminyl-11-(3-(4-pyridin-3-yl-imidazol-1-yl)-propyl)hydrazo-9-methoxyimino-6-O-methyl-3-oxoerythronolide A, 11,12-carbamate

To a solution of 9-Deoxo-11-deoxy-5-O-desosaminyl-11-hydrazo-9-methoxyimino-6-O-methyl-3-oxoerythronolide A, 11,12-carbamate (300 mg, 0.45 mmod in toluene (4.0 mL) was added 3-(4-pyridin-3-yl-imidazol-1-yl)-propioaldehyde (100 mg, 0.50 mmol, 1.1 eq) and the reaction mixture was heated at 90° C. for 18 hours. Toluene was removed in vacuo and the residue was dissolved in MeOH (4.0 mL). HOAc (0.39 mL, 6.8 mmol) and NaBH₃CN (427 mg, 6.8 mmol) were added and the resulting solution was stirred at room temperature for 14 hours. MeOH was evaporated in vacuo, saturated NaHCO₃ was added to the residue, the aqueous layer was extracted with CH₂Cl₂ (3 times), the combined organic layers were washed with brine, dried over anhydrous MgSO₄, and concentrated in vacuo. The crude product was purified by preparative TLC (10% MeOH-1% NH₃.H₂O-89% CH₂Cl₂) to afford the title compound as a white solid.

¹H NMR (400 MHz, CDCl₃) δ: 8.94 (1H, s), 8.42 (1H), 8.03 (1H), 7.58 (1H, s), 7.36 (1H, s), 7.25 (1H), 6.11 (1H, s), 3.69 (3H, s), 2.64 (3H, s), 2.23 (6H, s), 1.45 (3H, s), 1.36 (3H, s), 1.30 (3H, d, J=5.6 Hz), 1.26 (3H, d, J=6.8 Hz), 1.21 (3H, d, J=4.8 Hz), 1.09 (3H, d, J=6.8Hz), 0.97 (3H, d, J=6.4 Hz), and 0.80 (3H, t, J=6.4 Hz).

¹³C NMR (100 MHz, CDCl₃) δ: 203.41, 170.01, 167.63, 156.73, 147.56, 146.40, 138.94, 138.13, 131.85, 130.31, 123.47, 115.12, 103.99, 81.36, 79.51, 78.51, 77.27, 70.33, 69.58, 65.84, 61.30, 59.24, 51.06, 50.40, 47.37, 44.39, 44.35, 40.23 (2C), 38.21, 33.86, 29.28, 28.12, 26.48, 22.11, 21.18, 19.91, 18.97, 17.45, 15.19, 14.49, 14.33, and 10.53.

MS: m/z 842 (M+H).

### EXAMPLE 12

### 9-Deoxo-11-deoxy-5-O-desosaminyl-11-(3-(imidazo(4,5-b)pyridin-3-yl)-propyl)hydrazo-9-methoxyimino-6-O-methyl-3-oxoerythronolide A, 11,12-carbamate

To a solution of 9-Deoxo-11-deoxy-5-O-desosaminyl-11-hydrazo-9-methoxyimino-6-O-methyl-3-oxoerythronolide A, 11,12-carbamate (2.0 g, 3.05 mmol) in toluene (30 mL) was added 3-(imidazo(4,5-b)pyridin-3-yl)-propioaldehyde (910 mg, 4.26 mmol, 1.4 eq) and the reaction mixture was heated at 90° C. for 18 hours. Toluene was removed in vacuo and the residue was dissolved in MeOH (30 mL). HOAc (2.8 mL, 48.72 mmol) and NaBH₃CN (1.91 g, 30.45 mmol) were added and the resulting solution was stirred at room temperature for 14 hours. MeOH was evaporated in vacuo, saturated NaHCO₃ was added to the residue, the aqueous layer was extracted with CH₂Cl₂ (3 times), the combined organic layers were washed with brine, dried over anhydrous MgSO₄, and concentrated in vacuo. The crude product was purified by preparative TLC (10% MeOH-1% NH₃.H₂O-89% CH₂Cl₂) to afford the title compound as a white solid.

¹H NMR (400 MHz, CDCl₃) δ: 8.35 (1H), 8.22 (1H, s), 8.02 (1H), 7.20 (1H), 7.36 (1H, s), 6.10 (1H, br t), 3.47 (3H, s), 2.62 (3H, s), 2.24 (6H, s), 1.46 (3H, s), 1.37 (3H, s), 1.30 (3H, d, J=6.8 Hz), 1.27 (3H, d, J=7.6 Hz), 1.22 (3H, d, J=6.0 Hz), 1.12 (3H, d, J=6.8 Hz), 0.99 (3H, d, J=7.2 Hz), and 0.84 (3H, t, J=7.6 Hz).

MS: m/z 816 (M+H).

### EXAMPLE 13

### 9-Deoxo-11-deoxy-5-O-desosaminyl-11-(3-(4-phenyl-imidazol-1-yl)-propyl)hydrazo-9-methoxyimino-6-O-methyl-3-oxoerythronolide A, 11,12-carbamate

To a solution of 9-Deoxo-11-deoxy-5-O-desosaminyl-11-hydrazo-9-methoxyimino-6-O-methyl-3-oxoerythronolide A, 11,12-carbamate (1.37 g, 2.09 mmol) in toluene (14 mL) was added 3-(4-phenyl-imidazol-1-yl)-propioaldehyde (583 mg, 2.92 mmol, 1.4 eq) and the reaction mixture was heated at 90° C. for 18 hours. Toluene was removed in vacuo and the residue was dissolved in MeOH (20 mL). HOAC (1.8 mL, 31.35 mmol) and NaBH₃CN (1.97 g, 31.35 mmol) were added and the resulting solution was stirred at room temperature for 14 hours. MeOH was evaporated in vacuo, saturated NaHCO₃ was added to the residue, the aqueous layer was extracted with CH₂Cl₂ (3 times), the combined organic layers were washed with brine, dried over anhydrous MgSO₄, and concentrated in vacuo. The crude product was purified by preparative TLC (10% MeOH-1% NH₃.H₂O-89% CH₂Cl₂) to afford the title compound as a white solid.

¹H NMR (400 MHz, CDCl₃) δ: 8.35 (1H), 8.22 (1H, s), 8.02 (1H), 7.20 (1H), 7.36 (1H, s), 6.10 (1H, br t), 3.69 (3H, s), 2.66 (3H, s), 2.26 (6H, s), 1.46 (3H, s), 1.37 (3H, s), 1.32 (3H, d, J=6.8 Hz), 1.27 (3H, d, J=7.6 Hz), 1.22 (3H, d, J=6.0 Hz), 1.11 (3H, d, J=7.2 Hz), 0.98 (3H, d, J=7.2 Hz), and 0.83 (3H, t,J=7.2 Hz).

MS: m/z 841 (M+H).

### EXAMPLE 14

### 9-Deoxo-11-deoxy-5-O-desosaminyl-10-epi-11-hydrazo-9-benzoxyimino-6-O-methyl-3-oxoerythronolide A, 11,12-carbamate

To a solution of 11-deoxy-5-O-desosaminyl-10-epi-11-hydrazo-6-O-methyl-3-oxoerythronolide A, 11,12-carbamate (202 mg, 0.322 mmol) in MeOH (3.0 mL) was added NH₂OBn.HCl (225 mg, 1.41 mmol, 4.4 eq) and the reaction mixture was heated at 72° C. for 16 hours. MeOH was removed in vacuo and the residue was dissolved in CH₂Cl₂. Saturated NaHCO₃ was added, the aqueous layer was extracted with CH₂Cl₂ (3 times), the combined organic layers were washed with brine, dried over anhydrous MgSO₄, and concentrated in vacuo. The crude product was purified by preparative TLC (10% MeOH-1% NH₃.H₂O-89% CH₂Cl₂) to afford the title compound as a white solid.

¹H NMR (400 MHz, CDCl₃) δ: 7.25 (5H, m), 5.39 (4H, br s), 5.09 (1H, d, J=12.8 Hz, AB), 4.99 (1H, d, J=12.8 Hz, AB), 4.93 (1H, dd, J=3.2, 9.2 Hz), 4.30 (1H, d, J=7.2 Hz), 3.98 (1H, d, J=10.8 Hz), 3.54 (1H, q, J=6.8 Hz), 3.9-1.3 (m), 2.75 (3H, s), 2.27 (6H, s), 2.02 (3H, d, J=6.8 Hz), 1.30 (3H, s), 1.27 (3H, d, J=7.2 Hz), 1.22 (3H, s), 1.21 (3H, d, J=6.8 Hz), 1.20 (3H, d, J=6.8 Hz), 1.09 (3H, d, J=7.2 Hz), and 0.814 (3H, t, J=7.6 Hz).

MS: m/z 733 (M+H).

### EXAMPLE 15

### 9-Deoxo-11-deoxy-5-O-desosaminyl-10-epi-11-hydrazo-9-hydroxyimino-6-O-methyl-3-oxoerythronolide A, 11,12-carbamate

To a solution of 11-deoxy-5-O-desosaminyl-10-epi-11-hydrazo-6-O-methyl-3-oxoerythronolide A, 11,12-carbamate (499 mg, 0.795 mmol) in i-PrOH (7.0 mL) was added NH₂OH.HCl (579 mg, 8.33 mmol, 10.5 eq) and the reaction mixture was heated at 80° C. for three days. i-PrOH was removed in vacuo and the residue was dissolved in CH₂Cl₂. Saturated NaHCO₃ was added, the aqueous layer was extracted with CH₂Cl₂ (3 times), the combined organic layers were washed with brine, dried over anhydrous MgSO₄, and concentrated in vacuo. The crude product was purified by preparative TLC (10% MeOH-1% NH₃.H₂O-89% CH₂Cl₂) to afford a 3:2 mixture of the title compound as a white solid (157 mg, 31%).

¹H NMR (400 MHz, CDCl₃) of the major isomer, δ: 2.77 (3H, s, 6-OMe), 2.26 (6H, s, NMe₂), 0.84 (3H, t, J=7.0 Hz, 15-Me); MS: m/z 610 (M+H).

¹H NMR (400 MHz, CDCl₃) of the minor isomer, δ: 2.68 (3H, s, 6-OMe), 2.25 (6H, s, NMe₂), 0.81 (3H, t, J 7.0 Hz, 15-Me); MS: m/z 610 (M+H).

### EXAMPLE 16

### 9-Deoxo-11-deoxy-5-O-desosaminyl-11-hydrazo-9-hydroxyimino-6-O-methyl-3-oxoerythronolide A, 11,12-carbamate

To a solution of 11-deoxy-5-O-desosaminyl-11-hydrazo-6-O-methyl-3-oxoerythronolide A, 11,12-carbamate (20 g, 31.8 mmol) in EtOH (210 mL) was added NH₂OH-HCl (33.1g, 477 mmol, 15 eq) and pyridine (38.4 mL, 477 mmol, 15 eq), and the resulting solution at 80° C. for 38 h. EtOH was removed in vacuo and the residue was dissolved in CH₂Cl₂. Saturated NaHCO₃ was added, the aqueous layer was extracted with CH₂Cl₂ (3 times), the combined organic layers were washed with brine, dried over anhydrous MgSO₄, and concentrated in vacuo. The crude product was purified by silica gel flash chromatography (3% MeOH-0.3% NH₃.H₂O-96.7% CH₂Cl₂) to afford the title compound as a white solid (7.3g).

¹H NMR (400 MHz, CDCl₃) δ: 9.81 (1H, br. s), 2.67 (3H, s), 2.57 (1H, q, J=6.8 Hz), 2.25 (6H, s), 1.47 (3H, s), 1.43 (3H, s), 1.32 (3H, d, J=6.8 Hz), 1.25 (3H, d, J=7.6 Hz), 1.21 (3H, d, J=6.4 Hz), 1.11 (3H, d, J=6.8 Hz), 0.97 (3H, d, J=7.2 Hz), 0.83 (3H, t, J=7.2Hz).

¹³H NMR (100 MHz, CDCl₃) δ: 203.81, 169.80, 165.97, 156.68, 103.91, 81.53, 79.85, 78.49, 76.74, 70.35, 69.48, 65.85, 64.33, 51.10, 49.66, 47.73, 40.24, 38.42, 33.73, 28.19, 25.55, 22.11, 21.15, 19.98, 18.64, 16.96, 15.68, 14.25, 13.71, and 10.39.

MS: m/z 643 (M+H).

### EXAMPLE 17

### 9-Deoxo-11-deoxy-5-O-desosaminyl-11-(3-quinolin-4-yl-propylidene)hydrazo-9-hydroxyimino-6-O-methyl-3-oxoerythronolide A, 11,12-carbamate

To a solution of 9-deoxo-11-deoxy-5-O-desosaminyl-11-hydrazo-9-hydroxyimino-6-O-methyl-3-oxoerythronolide A, 11,12-carbamate (150 mg, 0.23 mmol) in anhydrous toluene (2.3 mL) was added 3-(4-quinolinyl)propionaldehyde (85 mg, 0.46 mmol) and the resulting solution was heated at 90° C. for 18 h. EtOH was evaporated in vacuo and the crude product was purified by silica gel flash chromatography (5% MeOH-0.5% NH₃.H₂094.5% CH₂Cl₂) to afford the title compound.

¹H NMR (400 MHz, CDCl₃) δ: 2.84 (3H, s), 2.30 (6H, s), 1.58 (3H, s), 1.54 (3H, s), 1.34 (3H, d, J=6.8 Hz), 1.32 (3H, d, J=7.2 Hz), 1.28 (3H, d, J 6.0 Hz), 1.25 (3H, d, J=6.0 Hz), 1.05 (3H, d, J=7.2 Hz), 0.86 (3H, t, J 7.2 Hz).

MS: m/z 810 (M+H).

### EXAMPLE 18

### 9-Deoxo-11-deoxy-5-O-desosaminyl-11-(3-benzoimidazol-4-yl-propyl)hydrazo-9-hydroxyimino-6-O-methyl-3-oxoerythronolide A, 11,12-carbamate

To a solution of 9-deoxo-11-deoxy-5-O-desosaminyl-11-hydrazo-9-hydroxyimino-6-O-methyl-3-oxoerythronolide A, 11,12-carbamate (100 mg, 0.16 mmol) in anhydrous toluene (1.6 mL) was added 3-(benzoimidazol-1-yl)propionaldehyde (62 mg, 0.36 mmol) and the resulting solution was heated at 90° C. for 18 h. EtOH was evaporated in vacuo and the crude product was dissolved in methanol (1.5 mL). To this solution was added HOAc (137uL, 2.4 mmol) and NaBH₃CN (103 mg, 1.5 mmol) and the resulting mixture was stirred at room temperature for 12 h. MeOH was removed in vacuo and the residue was dissolved in CH₂Cl₂. Saturated NaHCO₃ was added, the aqueous layer was extracted with CH₂Cl₂ (3 times), the combined organic layers were washed with brine, dried over anhydrous MgSO₄, and concentrated in vacuo. The crude product was purified by preparative TLC (10% MeOH-1% NH₃.H₂O-89% CH₂Cl₂) to afford the title compound as a white solid (57 mg, 46%).

¹H NMR (400 MHz, CDCl₃) δ: 11. 00 (1-H, br s), 2.71 (3H, s), 2.30 (6H, s), 1.49 (3H, s), 1.47 (3H, s), 1.34 (3H, d, J=6.8 Hz), 1.28 (3H, d, J=7.6 Hz), 1.23 (3H, d, J=6.0 Hz), 1.11 (3H, d, J=7.2 Hz), 0.99 (3H, d, J=6.8 Hz), and 0.824 (3H, t, J=7.2 Hz). 2.84 (3H, s), 2.30 (6H, s), 1.58 (3l, s), 1.54 (3H, s), 1.34 (3H, d, J=6.8 Hz), 1.32 (3H, d, J=7.2 Hz), 1.28 (3H, d, J=6.0 Hz), 1.25 (3H, d, J=6.0 Hz), 1.05 (3H, d, J=7.2 Hz), 0.86 (3H, t, J=7.2 Hz).

¹³H NMR (100 MHz, CDCl₃) δ: 203.45, 169.92, 166.21, 156.74, 143.31, 142.85, 133.53, 122.90, 122.12, 119.47, 110.23, 103.93, 81.33, 79.87, 78.59, 77.24, 70.29, 69.38, 65.97, 59.68, 51.09, 50.62, 47.45, 44.45, 42.34, 40.26, 38.23, 33.59, 28.49, 27.40, 25.55, 22.18, 21.15, 20.10, 19.11, 17.32, 15.28, 14.53, 14.39, and 10.51.

MS: m/z 801 (M+H).

The following compounds were prepared from 9-deoxo-11-deoxy-5-O-desosaminyl-11 hydrazo-9-hydroxyimino-6-O-methyl-3-oxoerythronolide A, 11,12-carbamate and appropriate aldehydes by using the procedures as described above.

### EXAMPLE 19

### 9-Deoxo-11-deoxy-5-O-desosaminyl-11-(3-(4-phenyl-imidazol-1-yl)-propyl)hydrazo-9-hydroxyimino-6-O-methyl-3-oxoerythronolide A, 11,12-carbamate

¹H NMR (400 MHz, CDCl₃) δ: 2.69 (3H, s), 2.29 (6H, s), 1.49 (3H, s), 1.47 (3H, s), 1.32 (3H, d, J=6.8 Hz), 1.27 (3H, d, J=7.6 Hz), 1.24 (3H, d, J=6.0 Hz), 1.10 (3H, d, J=6.8 Hz), 1.00 (3H, d, J=6.8 Hz), and 0.83 (3H, t, J=7.2 Hz).

MS: m/z 801 (M+H).

### EXAMPLE 20

### 9-Deoxo-11-deoxy-5-O-desosaminyl-11-(3-benzotrizol-2-yl-propyl)hydrazo-9-hydroxyimino-6-O-methyl-3-oxoerythronolide A, 11,12-carbamate

¹H NMR (400 MHz, CDCl₃) δ: 9.95 (1H, br. s), 1.44 (3H, s), 1.39 (3H, s), 1.25 (6H, d, J=6.8 Hz), 1.23 (3H, d, J=6.0 Hz), 1.09 (3H, d, J=6.8 Hz), 1.03 (3H, d, J=6.8 Hz), and 0.81 (3H, t, J=7.6 Hz).

MS: m/z 802 (M+H).

### EXAMPLE 21

### 9-Deoxo-11-deoxy-5-O-desosaminyl-11-(3-benzotrizol-1-yl-propyl)hydrazo-9-hydroxyimino-6-O-methyl-3-oxoerythronolide A, 11,12-carbamate

¹H NMR (400 MHz, CDCl₃) δ: 9.02 (1H, br. s), 6.23 (1H, br. s), 2.58 (3H, s), 2.32 (6H, s), 1.47 (6H, s), 1.29 (3H, d, J=6.8 Hz), 1.26 (3H, d, J=7.6 Hz), 1.23 (3H, d, J=6.0 Hz), 1.10 (3H, d, J=6.8 Hz), 1.01 (3H, d, J=6.8 Hz), and 0.81 (3H, t, J=7.2 Hz).

MS: m/z 802 (M+H).

### EXAMPLE 22

### 9-Deoxo-11-deoxy-5-O-desosaminyl-11-(3-phenylpropyl)hydrazo-9-hydroxwimino-6-O-methyl-3-oxoerythronolide A, 11,12-carbamate

¹H NMR (400 MHz, CDCl₃) δ: 9.59 (1H, br. s), 7.10 (5H, m), 6.55 (1H, br. s), 2.67 (3H, s), 2.24 (6H, s), 1.43 (6H, s), 1.33 (3H, d, J=6.8 Hz), 1.25 (3H, d, J=7.2 Hz), 1.21 (3H, d, J=6.0 Hz), 1.00 (3H, d, J=6.8 Hz), 0.92 (3H, d, J=6.4 Hz), and 0.82 (3H, t, J=7.2 Hz).

¹³C NMR (100 MHz, CDCl₃) δ: 203.70, 169.59, 166.73, 156.24, 142.12, 128.42 (2C), 128.20 (2C), 125.62, 103.94, 81.35, 79.63, 78.56, 77.11, 70.36, 69.48, 65.87, 59.50, 51.13, 50.40, 48.02, 47.52, 40.24 (2C), 38.21, 33.60, 33.14, 29.77, 28.21, 25.57, 22.19, 21.15, 20.13, 19.05, 17.13, 15.41, 14.46, 14.29, and 10.49.

MS: m/z 761 (M+H).

### EXAMPLE 23

### 9-Deoxo-1-deoxy-5-O-desosaminyl-11-(3-(4-hydroxyphenyl)-propyl)hydrazo-9-hydroxyimino-6-O-methyl-3-oxoerythronolide A, 11,12-carbamate

¹H NMR (400 MHz, CDCl₃) δ: 6.91 (2H), 6.68 (2H), 6.43 (1H, br. s), 2.67 (3H, s), 2.30 (6H, s), 1.44 (3H, s), 1.43 (3H, s), 1.33 (3H, d, J=6.8 Hz), 1.25 (3H, d, J=7.2 Hz), 1.21 (3H, d, J=6.0 Hz), 1.01 (3H, d, J=6.8 Hz), 0.92 (3H, d, J=6.4 Hz), and 0.80 (3H, t, J=7.2 Hz).

MS: m/z 778 (M+H).

### EXAMPLE 24

### 9-Deoxo-11-deoxy-5-O-desosaminyl-11-(3-(4-methoxyipheny)-propyl)hydrazo-9-hydroxyimino-6-O-methyl-3-oxoerythronolide A, 11,12-carbamate

¹H NMR (400 MHz, CDCl₃) δ: 9.38 (1H, br. s), 7.04 (2H, d, J=8.4 Hz), 6.74 (2H, d, J=8.4 Hz), 6.44 (1H, br. s), 3.74 (3H, s), 2.67 (3H, s), 2.30 (6H, s), 1.44 (6H, s), 1.33 (3H, d, J=6.8 Hz), 1.25 (3H, d, J=7.6 Hz), 1.22 (3H, d, J=6.0 Hz), 1.01 (3H, d, J=6.8 Hz), 0.93 (3H, d, J=6.8 Hz), and 0.82 (3H, t, J=7.2 Hz).

¹³H NMR (100 MHz, CDCl₃) δ: 203.71, 169.58, 167.06, 157.64, 156.30, 134.23, 129.29 (2C), 113.68 (2C), 103.84, 81.33, 79.61, 78.51, 77.16, 70.27, 69.34, 65.98, 59.51, 55.19, 51.12, 50.44, 47.95, 47.45, 40.26 (2C), 38.19, 33.64, 32.29, 30.06, 28.46, 25.55, 22.19, 21.12, 20.09, 19.05, 17.17, 15.34, 14.49, 14.33, and 10.49.

MS: m/z 792 (M+H).

### EXAMPLE 25

### 9-Deoxo-11-deoxy-5-O-desosaminyl-11-(3-(2-methoxyphenyl)-propyl)hydrazo-9-hydroxyimino-6-O-methyl-3-oxoerythronoride A, 11,12-carbamate

¹H NMR (400 MHz, CDCl₃) δ: 8.66 (1H, br. s), 7.13 (2H), 6.82 (2H), 3.80 (3H, s), 2.68 (3H, s), 2.30 (6H, s), 1.45 (3H, s), 1.43 (3H, s), 1.32 (3H, d, J=6.8 Hz), 1.26 (3H, d, J=7.6 Hz), 1.22 (3H, d, J=6.4 Hz), 1.06 (3H, d, J=6.8-Hz), 0.98 (3H, d, J=6.4 Hz), and 0.83 (3H, t, J=7.2 Hz).

¹³H NMR (100 MHz, CDCl₃) δ: 203.78, 169.59, 167.39, 157.04, 156.14, 130.36, 129.29, 126.96, 120.82, 110.49, 103.86, 81.19, 79.16, 78.42, 77.24, 70.27, 69.37, 65.94, 59.88, 55.54, 51.10, 50.56, 48.26, 47.34, 40.24 (2C), 38.22, 33.70, 28.44, 27.83, 27.45, 25.58, 22.19, 21.13, 20.01, 19.07, 17.29, 15.21, 14.50, 14.43, and 10.37.

MS: m/z 792 (M+H).

### EXAMPLE 26

### 9-Deoxo-11-deoxy-5-O-desosaminyl-11-(benzyl)hydrazo-9-hydroxyimino-6-O-methyl-3-oxoerythronolide A, 11,12-carbamate

¹H NMR (400 MHz, CDCl₃) δ: 7.95 (1H, br. s), 6.23 (1H, br. s), 2.88 (3H, s), 2.29 (6H, s), 1.51 (3H, s), 1.44 (3H, s), 1.36 (3H, d, J=6.8 Hz), 1.29 (3H, d, J=7.6 Hz), 1.25 (3H, d, J=6.0 Hz), 1.05 (3H, d, J=6.8 Hz), 0.97 (3H, d, J=7.2 Hz), and 0.75 (3H, t, J=7.6 Hz).

MS: m/z 733 (M+H).

### EXAMPLE 27

### 9-Deoxo-11-deoxy-5-O-desosaminyl-11-(pyridin-4-yl-methyl)hydrazo-9-hydroxyimino-6-O-methyl-3-oxoerythronolide A, 11,12-carbamate

¹H NMR (400 MHz, CDCl₃) δ: 8.07 (2H), 7.25 (2H), 6.39 (1H, br. s), 2.87 (3H, s), 2.27 (6H, s), 1.54 (3H, s), 1.46 (3H, s), 1.34 (3H, d, J=6.8 Hz), 1.29 (3H, d, J=7.2 Hz), 1.24 (3H, d, J=6.0Hz), 1.13 (3H, d, J=6.8 Hz), 1.01 (3H, d, J=7.2Hz), 0.76 (3H, t, J=7.2 Hz).

¹³C NMR (100 MHz, CDCl₃) δ: 203.72, 169.91, 166.50, 155.76, 148.16 (2C), 147.70, 123.71 (2C), 103.99, 81.17, 79.75, 78.71, 77.26, 70.34, 69.50, 65.92, 60.30, 51.69, 51.15, 50.74, 47.62, 40.26 (2C), 38.25, 33.69, 28.29, 25.54, 22.02, 21.16, 20.16, 19.14, 17.36, 15.42, 14.50, 14.29, and 10.27.

MS: m/z 733 (M+H).

### EXAMPLE 28

### 9-Deoxo-11-deoxy-5-O-desosaminyl-11-3-(pyridin-4-yl-propyl)hydrazo-9-hydroxyimino-6-O-methyl-3-oxoerythronolide A, 11,12-carbamate

¹H NMR (400 MHz, CDCl₃) δ: 11.62 (1H, s), 8.24 (2H), 6.91 (2H), 6.20 (1H, br. s), 2.76 (3H, s), 2.31 (6H, s), 1.51 (3H, s), 1.46 (3H, s), 1.33 (3H, d, J=6.4 Hz), 1.27 (3H, d, J=7.2 Hz), 1.23 (3H, d, J=6.0 Hz), 1.10 (3H, d, J=6.8 Hz), 1.00 (3H, d, J=6.8 Hz), 0.83 (3H, t, J=7.2 Hz).

13C NMR (100 MHz, CDCl₃) δ: 203.71, 169.78, 166.30, 156.35, 152.39, 140.45, 124.10 (2C), 124.10 (2C), 103.86, 3, 79.71, 78.59, 77.24, 70.27, 69.34, 65.98, 59.50, 51.15, 50.44, 47.55 (2C), 40.27 (2C), 38.21, 33.60, 32.50, 28.54, 27.90, 25.51, 22.14, 21.13, 20.11, 19.11, 17.33, 15.42, 14.48, 14.32, and 10.44.

MS: m/z 762(M+H).

### EXAMPLE 29

### 9-Deoxo-11-deoxy-5-O-desosaminyl-11-3-(pyridin-3-yl-propyl)hydrazo-9-hydroxyimino-6-O-methyl-3-oxoerythronolide A, 11,12-carbamate

1H NMR (400 MHz, CDCl₃) δ: 10.96 (1H, s), 8.30 (1KH, d, J=4.4 Hz), 8.21 (1H, s), 7.40 (1H, d, J=8.0 Hz), 7.10 (1I, dd, J=5.2 and 7.6 Hz), 6.16 (1H, br. s), 2.70 (3K, s), 2.27 (6H, s), 1.48 (3H, s), 1.44 (3H, s), 1.31 (3H, d, J=7.2 Hz), 1.25 (3H, d, J=7.6 Hz), 1.21 (3H, d, J=6.0 Hz), 1.06 (3H, d, J=6.8 Hz), 0.96 (3H, d, J=6.8 Hz), 0.80 (3H, t, J=7.6 Hz).

MS: m/z 762(M+H).

### EXAMPLE 30

### 9-Deoxo-11-deoxy-5-O-desosaminyl-11-(phenylethyl)hydrazo-9-hydroxyimino-6-O-methyl-3-oxoerythronolide A, 11,12-carbamate

¹H NMR (400 MHz, CDCl₃) δ: 7.25 (5H, m), 2.59 (3H, s), 2.28 (6H, s), 1.40 (3H, s), 1.32 (3H, d, J=6.8 Hz), 1.30 (3H, s), 1.25 (3H, d, J=7.6 Hz), 1.22 (3H, d, J=6.0 Hz), 0.98 (3H, d, J=6.8 Hz), 0.88 (3H, d, J=6.8 Hz), 0.83 (3H, t, J=7.6 Hz).

MS: m/z 747 (M+H).

### EXAMPLE 31

### 9-Deoxo-11-deoxy-5-O-desosaminyl-11-(3-(4-methoxyphenyl)-(1,2,4)oxadizol-5-yl)-propyl)hydrazo-9-hydroxyimino-6-O-methyl-3-oxoerythronolide A, 11,12-carbamate

¹H NMR (400 MHz, CDCl₃) δ: 9.65 (1H, br. s), 7.87 (2H, d, J=8.4 Hz), 6.92 (2H, d, J=9.2 Hz), 6.16 (1H, br. s), 3.80 (3H, s), 2.46 (3H, s), 2.24 (6H, s), 1.03 (3H, d, J=7.2 Hz), 0.96 (3H, d, J=7.2 Hz), and 0.81 (3H. t, J=7.2 Hz).

MS: m/z 859 (M+H).

### EXAMPLE 32

### 9-Deoxo-11-deoxy-5-O-desosaminyl-11-(3-(4-chlorophenyl)-(1,2,4)oxadizol-5-yl-propyl)hydrazo-9-hydroxyimino-6-O-methyl-3-oxoerythronolide A, 11,12-carbamate

¹H NMR (400 MHz, CDCl₃) δ: 7.89 (2H, d, J=8.8 Hz), 7.39 (2H, d, J=8.8 Hz), 6.20 (1H, br. s), 2.49 (3H, s), 2.24 (6H, s), 1.42 (3H, s), 1.03 (3H, d, J=7.2 Hz), 0.96 (3H, d, J=6.8 Hz), and 0.82 (3H, t, J=7.2 Hz).

MS: m/z 863 (M+H).

### EXAMPLE 33

### 9-Deoxo-11-deoxy-5-O-desosaminyl-11-(3-indol-1-yl-propyl)hydrazo-9-hydroxyimino-6-O-methyl-3-oxoerythronolide A, 11,12-carbamate

¹H NMR (400 MHz, CDCl₃) δ: 8.63 (1H, br. s), 8.02 (1H), 7.28 (1H), 7.12 (1H), 7.04 (1H), 6.89 (1H), 6.23 (1H, br. s), 2.60 (3H, s), 2.30 (6H, s), 1.44 (3H, s), 1.39 (3H, s), 1.34 (3H, d, J=6.8 Hz), 1.25 (3H, d, J=7.6 Hz), 1.22 (3H, d, J=6.4 Hz), 1.02 (3H, d, J=7.2 Hz), 0.92 (3H, d, J=6.8 Hz), and 0.82 (3H, t, J=7.6 Hz).

MS: m/z 800 (M+H).

### EXAMPLE 34

### 9-Deoxo-11-deoxy-5-O-desosaminyl-11-(3-indazol-1-yl-propyl)hydrazo-9-hydroxyimino-6-O-methyl-3-oxoerythronolide A, 11,12-carbamate

¹H NMR (400 MHz, CDCl₃) δ: 10.53 (1H, br. s), 7.91 (1H), 7.68 (1H), 7.44 (1H), 7.26 (1H), 7.12 (1H), 6.40 (1H, br. s), 2.54 (3H, s), 2.25 (6H, s), 1.48 (3H, s), 1.44 (3H, s), 1.08 (3H, d, J=6.8 Hz), 1.02 (3H, d, J=7.2 Hz), and 0.79 (3H, t, J=7.2 Hz).

MS: m/z 801 (M+H).

### EXAMPLE 35

### 9-Deoxo-11-deoxy-5-O-desosaminyl-11-(3-(5-phenyl-1H-pyrrol-2-yl)-propyl)hydrazo-9-hydroxyimino-6-O-methyl-3-oxoerythronolide A, 11,12-carbamate

¹H NMR (400 MHz, CDCl₃) δ: 9.36 (1H, br. s), 7.49 (2H), 7.30 (2H), 7.10 (1H), 6.35 (1H), 5.88 (1H), 2.66 (3H, s), 2.25 (6H, s), 1.45 (3H, S), 1.38(3H, s), 1.32 (3H, d, J=6.8 Hz), 1.25 (3H, d, J=7.6 Hz), 1.18 (3H, d, J=6.4 Hz), 1.05 (3H, d, J=6.8 Hz), 0.93 (3H, d, J=7.2 Hz), and 0.72 (3H, t, J=7.2 Hz).

¹³H NMR (100 MHz, CDCl₃) δ: 203.54, 169.84, 167.07, 156.85, 133.90, 133.21, 130.84, 128.58, 125.11, 123.35, 106.83, 105.41, 103.89, 81.18, 79.60, 78.52, 77.17, 70.27, 69.37, 65.90, 59.84, 51.12, 50.52, 47.76, 47.49, 40.23, 38.17, 33.64, 28.56, 28.36, 25.57, 25.01, 22.22, 21.12, 20.00, 19.04, 17.19, 15.37, 14.48, 14.28, and 10.39.

MS: m/z 826 (M+H).

### EXAMPLE 36

### 9-Deoxo-11-deoxy-5-O-desosaminyl-11-(3-carbazol-9-yl-propyl)hydrazo-9-hydroxyimino-6-O-methyl-3-oxoerythronolide A, 11,12-carbamate

¹H NMR (400 MHz, CDCl₃) δ: 7.98 (2H), 7.46 (2H), 7.38 (2H), 7.12 (2H), 2.55 (3H, s), 2.23 (6H, s), 1.44 (3H, s), 1.40(3H, s), 1.35 (3H, d, J=7.2 Hz), 1.03 (3H, d, J=6.8 Hz), 0.94 (3H, d, J=6.8 Hz), and 0.76 (3H, t, J=7.2 Hz).

MS: m/z 851 (M+H).

### EXAMPLE 37

### 9-Deoxo-11-deoxy-5-desosaminyl-11-(3-(1H-indol-3-yl)-propyl)hydrazo-9-hydroxyimino-6-O-methyl-3-oxoerythronolide A, 11,12-carbamate

¹H NMR (400 MHz, CDCl₃) δ: 8.75 (1H, br. s), 7.98 (1H), 7.55 (1H), 7.25 (1H), 7.12 (1H), 7.05 (1H), 6.86 (1H), 6.21 (1H, br. s), 2.58 (3H, s), 2.26 (6H, s), 1.44 (3H, s), 1.39(3H, s), 1.34 (3H, d, J=6.0 Hz), 1.01 (3H, d, J=6.4 Hz), 0.92 (3H, d, J=6.8 Hz), and 0.81 (3H, t, J=7.2 Hz).

MS: m/z 800 (M+H).

### EXAMPLE 38

### 9-Deoxo-11-deoxy-5-O-desosaminyl-11-(3-furan-2-yl-propyl)hydrazo-9-hydroxyimino-6-O-methyl-3-oxoerythronolide A, 11,12-carbamate

¹H NMR (400 MHz, CDCl₃) δ: 9.18 (1H, br. s), 7.21 (1H, s), 5.93 (1H, s), 6.42 (1H, br. s), 5.93 (1H, s), 2.68 (3H, s), 2.25 (6H, s), 1.45 (3H, s), 1.44 (3H, s), 1.32 (3H, d, J=6.4 Hz), 1.26 (3H, d, J=7.2 Hz), 1.21 (3H, d, J=6.4 Hz), 1.01 (3H, d, J=6.8 Hz), 0.93 (3H, d, J=6.8 Hz), and 0.83 (3H, t, J=7.2Hz).

¹³H NMR (100 MHz, CDCl₃) δ: 203.71, 169.61, 167.70, 156.30, 155.74, 140.73, 110.02, 104.94, 103.94, 81.34, 79.65, 78.51, 77.11, 70.34, 69.51, 65.86, 59.55, 51.11, 50.44, 47.61, 47.50, 40.25 (2C), 38.20, 33.60, 28.16, 26.22, 25.58, 25.22, 22.19, 21.17, 20.14, 19.07, 17.13, 15.39, 14.47, 14.33, and 10.47.

MS: m/z 751 (M+H).

### EXAMPLE 39

### 9-Deoxo-11-deoxy-5-O-desosaminyl-11-(3-pvrrol-1-yl-propyl)hydrazo-9-hydroxyimino-6-O-methyl-3-oxoerythronolide A, 11,12-carbamate

¹H NMR (400 MHz, CDCl₃) δ: 8.51 (1H, br. s), 7.21 (1H, s), 6.64 (1H), 6.05 (1H), 2.68 (3H, s), 2.33 (6H, s), 1.45 (3H, s), 1.43 (3H, s), 1.32 (3H, d, J=6.4 Hz), 1.26 (3H, d, J=7.2 Hz), 1.23 (3H, d, J=6.0 Hz), 1.01 (3H, d, J=6.8 Hz), 0.93 (3H, d, J=7.2 Hz), and 0.84 (3H, t, J=7.2 Hz).

MS: m/z 750 (M+H).

### EXAMPLE 40

### 9-Deoxo-11-deoxy-5-O-desosaminvI-11-(3-pyrazol-1-yl-propyl)hydrazo-9-hydroxyimino-6-O-methyl-3-oxoerythronolide A, 11,12-carbamate

¹H NMR (400 MHz, CDCl₃) δ: 10.83 (1H, br. s), 7.40 (1H), 7.28 (1H), 6.20 (1H), 6.05 (1H), 2.52 (3H, s), 2.24 (6H, s), 1.45 (3H, s), 1.43 (3H, s), 1.28 (3H, d, J=6.4 Hz), 1.24 (3H, d, J=7.6 Hz), 1.19 (3H, d, J=6.4 Hz), 1.05 (3H, d, J=6.8 Hz), 0.99 (3H, d, J=6.8 Hz), and 0.80 (3H, t, J=7.2Hz).

MS: m/z 751 (M+H).

### EXAMPLE 41

### 9-Deoxo-11-deoxy-5-O-desosaminyl-11-(3-naphthalen-1-yl-propyl)hydrazo-9-hydroxyimino-6-O-methyl-3-oxoerythronolide A, 1,12-carbamate

¹H NMR (400 MHz, CDCl₃) δ: 9.32 (1H, br. s), 8.04 (1H), 7.76 (1H), 7.61 (1H), 7.40 (2H), 7.24 (2H), 2.65 (3H, s), 2.31 (6H, s), 1.44 (3H, s), 1.42 (3H, s), 1.34 (3H, d, J=6.8 Hz), 1.25 (3H, d, J=7.6 Hz), 1.23 (3H, d, J=6.0 Hz), 1.02 (3H, d, J=6.8 Hz), 0.94 (3H, d, J=6.8 Hz), and 0.79 (3H, t, J=7.6 Hz).

MS: m/z 811 (M+H).

The following compounds can be prepared from 9-deoxo-11-deoxy-5-O-desosaminyl-11-hydrazo-9-hydroxyimino-6-O-methyl-3-oxoerythronolide A, 11,12-carbamate and appropriate aldehydes by using the procedures as described as above.

9-Deoxo-11-deoxy-5-O-desosaminyl-11-(3-(4-phenyl-1H-imidazol-2-yl)-propyl)hydrazo-9-hydroxyimino-6-O-methyl-3-oxoerythronolide A, 11,12-carbamate;

9-Deoxo-11-deoxy-5-O-desosaminyl-11-(3-(2-pyridin-3-yl-thiazol-4-yl)-propyl)hydrazo-9-hydroxyimino-6-O-methyl-3-oxoerythronolide A, 11,12-carbamate;

9-Deoxo-11-deoxy-5-O-desosaminyl-11-(3-(2-phenyl-thiazol-5-yl)-propyl)hydrazo-9-hydroxyimino-6-O-methyl-3-oxoerythronolide A, 11,12-carbamate;

9-Deoxo-11-deoxy-5-O-desosaminyl-11-(3-thiophen-2-yl-propyl)hydrazo-9-hydroxyimino-6-O-methyl-3-oxoerythronolide A, 11,12-carbamate;

9-Deoxo-11-deoxy-5-O-desosaminyl-11-(3-(7-methoxy-quinolin-4-yl)-propyl)hydrazo-9-hydroxyimino-6-O-methyl-3-oxoerythronolide A, 11,12-carbamate.

What is claimed is:

1. A compound of the formula 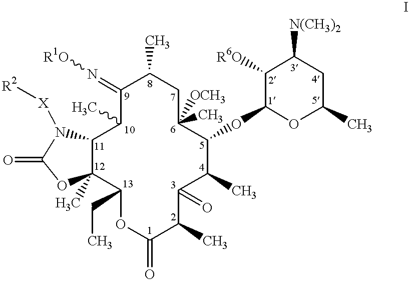
or a pharmaceutically acceptable salt thereof, wherein:
X is —CR⁷R⁸— or —NR⁷—;
or X is taken together with R² to form —N═CR⁴R⁵;
or X and R² are taken together to form a heterocyclic ring of the formula XVI: 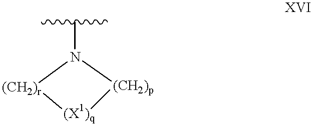
wherein in said ring of formula XVI, r and p are each independently an integer ranging from 1 to 3, q is 0 or 1, and X¹ is —CH₂—, O, S, —C(O)—, —C(S)—, —SO₂—, —CH═CH—, —CH(OH)CH(OH)—, or —NH—; and wherein the (CH₂)ᵣ and (CH₂)ₚ portions of said ring of formula XVI are optionally substituted by 1 to 4 substituents, and the nitrogen atom where X¹ is —NH— is optionally substituted by 1 substituent, said optional substituents being independently selected from the group consisting of —C(O)O(C₁-C₁₀ alkyl), C₁-C₁₀ alkoxy, C₁-C₁₀ alkanoyl, halo, nitro, cyano, 5-10 membered heterocyclyl, C₁-C₁₀ alkyl, —NR⁷R⁸, C₆-C₁₀ aryl, —S(O)ₙ(C₁-C₁₀ alkyl) wherein n is an integer ranging from 0 to 2, and —SO₂NR⁷R⁸;
R¹ is H or C₁-C₁₀ alkyl, wherein 1 to 3 carbons of said alkyl are optionally replaced by a heteroatom selected from O, S and N, and said alkyl is optionally substituted by 1 to 3 substituents independently selected from the group consisting of —C(O)O(C₁-C₁₀ alkyl), C₁-C₁₀ alkoxy, C₁-C₁₀ alkanoyl, halo, nitro, cyano, 5-10 membered heterocyclyl, C₁-C₁₀ alkyl, —NR⁷R⁸, C₆-C₁₀ aryl, —S(O)ₙ(C₁-C₁₀ alkyl) wherein n is an integer ranging from 0 to 2, and —SO₂NR⁷R⁸;
R² is (i) H, R⁴, —C(O)R⁴, —C(O)OR⁴ or —(CR⁷R⁸)ₘR³ when X is —NR⁷—, or (ii) H, R⁴, or —(CR⁷R⁸)ₘR³ when X is —CR⁷R⁸—, wherein for both (i) and (ii) m is an integer ranging from 0 to 6 and both R⁷ and R⁸ may vary for each iteration where m is greater than 1;
each R³ is independently C₆-C₁₀ aryl or 5-10 membered heterocyclyl, wherein said aryl and heterocyclyl groups are optionally substituted by 1 to 3 substituents independently selected from the group consisting of —C(O)O(C₁-C₁₀ alkyl), C₁-C₁₀ alkoxy, C₁-C₁₀ alkanoyl, halo, nitro, cyano, 5-10 membered heterocyclyl, C₆-C₁₀ aryl, C₁-C₁₀ alkyl, —NR⁷R⁸, —S(O)ₙ(C₁-C₁₀ alkyl) wherein n is an integer ranging from 0 to 2, and —SO₂NR⁷R⁸; and,
each R⁴ and R⁵ is independently selected from H and C₁-C₁₂ alkyl wherein one or two carbons of said alkyl are optionally replaced be a heteroatom selected from O, S and N, and wherein said alkyl is optionally substituted by 1 to 3 substituents independently selected from the group consisting of —C(O)O(C₁-C₁₀ alkyl), C₁-C₁₀ alkoxy, C₁-C₁₀ alkanoyl, halo, nitro, cyano, C₁-C₁₀ alkyl, —NR⁷R⁸; C₆-C₁₀ aryl, 5-10 membered heterocyclyl, —S(O)ₙ(C₁-C₁₀ alkyl) wherein n is an integer ranging from 0 to 2, and —SO₂NR⁷R⁸;
R⁶ is H, —C(O)R³ or C₁-C₁₈ alkanoyl, wherein in the alkyl portion of said alkanoyl one or two carbons optionally may be replaced by a heteroatom selected from O, S and N; and,
each R⁷ and R⁸ is independently H or C₁-C₆ alkyl.

2. The compound of claim 1 wherein R⁶ is H.

3. The compound of claim 1 wherein X is —NH—.

4. The compound of claim 3 wherein R² is H.

5. The compound of claim 1 wherein R¹ is H, benzyl, C₁-C₃ alkyl, or —CH₂O(CH₂)₂OCH₃.

6. The compound of claim 3 wherein R² is —(CH₂)ₘR³ wherein m and R³ are as defined in claim 1.

7. The compound of claim 6 wherein R³ is 5-10 membered heterocyclyl.

8. The compound of claim 7 wherein R³ is quinolin-4-yl, 4-phenyl-1-imidazol-1-yl, imidazo(4,5-b)pyridin-3-yl, 4-pyridin-3-yl-imidazol-1-yl and pyridin-3-yl.

9. The compound of claim 1 wherein said compound is selected from the group consisting of:
9-Deoxo-11-deoxy-5-O-desosaminyl-11-(3-(4-pyridin-3-yl-imidazol-1-yl)-propyl)hydrazo-9-hydroxyimino-6-O-methyl-3-oxoerythronolide A, 11,12-carbamate;
9-Deoxo-11-deoxy-5-O-desosaminyl-11-(3-(4-pyridin-3-yl-imidazol-1-yl)-propyl)hydrazo-9-methoxyimino-6-O-methyl-3-oxoerythronolide A, 11,12-carbamate;
9-Deoxo-11-deoxy-5-O-desosaminyl-11-(3-(imidazo(4,5-b)pyridin-3-yl)-propyl)hydrazo-9-hydroxyimino-6-O-methyl-3-oxoerythronolide A, 11,12-carbamate;
9-Deoxo-11-deoxy-5-O-desosaminyl-11-(3-(imidazo(4,5-b)pyridin-3-yl)-propyl)hydrazo-9-methoxyimino-6-O-methyl-3-oxoerythronolide A, 11,12-carbamate;
9-Deoxo-11-deoxy-5-O-desosaminyl-11-(3-quinolin-4-yl-propyl)hydrazo-9-hydroxyimino-6-O-methyl-3-oxoerythronolide A, 11,12-carbamate;
9-Deoxo-11-deoxy-5-O-desosaminyl-11-(3-quinolin-4-yl-propyl)hydrazo-9-methoxyimino-6-O-methyl-3-oxoerythronolide A, 11,12-carbamate;
9-Deoxo-11-deoxy-5-O-desosaminyl-11-(3-(7-methoxy-quinolin-4-yl)-propyl))hydrazo-9-hydroxyimino-6-O-methyl-3-oxoerythronolide A, 11,12-carbamate;
9-Deoxo-11-deoxy-5-O-desosaminyl-11-(3-(7-methoxy-quinolin-4-yl)-propyl))hydrazo-9-methoxyimino-6-O-methyl-3-oxoerythronolide A, 11,12-carbamate
9-Deoxo-11-deoxy-5-O-desosaminyl-11-(3-quinolin-4-yl-propylidene)hydrazo-9-hydroxyimino-6-O-methyl-3-oxoerythronolide A, 11,12-carbamate;
9-Deoxo-11-deoxy-5-O-desosaminyl-11-(3-quinolin-4-yl-propylidene)hydrazo-9-methoxyimino-6-O-methyl-3-oxoerythronolide A, 11,12-carbamate;
9-Deoxo-11-deoxy-5-O-desosaminyl-11-(3-quinolin-4-yl-propyl)hydrazo-9-benzoxyimino-6-O-methyl-3-oxoerythronolide A, 11,12-carbamate;
19-Deoxo-1-deoxy-5-O-desosaminyl-11-(3-benzoimidazol-1-yl-propyl)hydrazo-9-hydroxyimino-6-O-methyl-3-oxoerythronolide A, 11,12-carbamate;
19-Deoxo-1-deoxy-5-O-desosaminyl-11-(3-benzoimidazol-1-yl-propyl)hydrazo-9-methoxyimino-6-O-methyl-3-oxoerythronolide A, 11,12-carbamate;
9-Deoxo-11-deoxy-5-O-desosaminyl-11-(3-indol-1-yl-propyl)hydrazo-9-hydroxyimino-6-O-methyl-3-oxoerythronolide A, 11,12-carbamate;
9-Deoxo-11-deoxy-5-O-desosaminyl-11-(3-indol-1-yl-propyl)hydrazo-9-methoxyimino-6-O-methyl-3-oxoerythronolide A, 11,12-carbamate;
9-Deoxo-11-deoxy-5-O-desosaminyl-11-(3-indazol-1-yl-propyl)hydrazo-9-hydroxyimino-6-O-methyl-3-oxoerythronolide A, 11,12-carbamate;
9-Deoxo-11-deoxy-5-O-desosaminyl-11-(3-indazol-1-yl-propyl)hydrazo-9-methoxyimino-6-O-methyl-3-oxoerythronolide A, 11,12-carbamate;
9-Deoxo-11-deoxy-5-O-desosaminyl-11-(3-carbazol-1-yl-propyl)hydrazo-9-hydroxyimino-6-O-methyl-3-oxoerythronolide A, 11,12-carbamate;
9-Deoxo-11-deoxy-5-O-desosaminyl-11-(3-carbazol-1-yl-propyl)hydrazo-9-methoxyimino-6-O-methyl-3-oxoerythronolide A, 11,12-carbamate;
9-Deoxo-11-deoxy-5-O-desosaminyl-11-(3-(5-phenyl-1H-pyrrol-2-yl)-propyl)hydrazo-9-hydroxyimino-6-O-methyl-3-oxoerythronolide A, 11,12-carbamate;
9-Deoxo-11-deoxy-5-O-desosaminyl-11-(3-(5-phenyl-1H-pyrrol-2-yl)-propyl)hydrazo-9-methoxyimino-6-O-methyl-3-oxoerythronolide A, 11,12-carbamate;
9-Deoxo-11-deoxy-5-O-desosaminyl-11-(3-(4-phenyl-imidazol-1-yl)-propyl)hydrazo-9-hydroxyimino-6-O-methyl-3-oxoerythronolide A, 11,12-carbamate;
9-Deoxo-11-deoxy-5-O-desosaminyl-11-(3-(4-phenyl-imidazol-1-yl)-propyl)hydrazo-9-methoxyimino-6-O-methyl-3-oxoerythronolide A, 11,12-carbamate;
9-Deoxo-11-deoxy-5-O-desosaminyl-11-(3-(3-(4-chlorophenyl)-(1,2,4)oxadizol-5-yl)-propyl)hydrazo-9-hydroxyimino-6-O-methyl-3-oxoerythronolide A, 11,12-carbamate;
9-Deoxo-11-deoxy-5-O-desosaminyl-11-(3-(3-(4-chlorophenyl)-(1,2,4)oxadizol-5-yl)-propyl)hydrazo-9-methoxyimino-6-O-methyl-3-oxoerythronolide A, 11,12-carbamate;
19-Deoxo-1-deoxy-5-O-desosaminyl-11-(3-(3-(4-methoxyphenyl)-(1,2,4)oxadizol-5-yl)-propyl)hydrazo-9-hydroxyimino-6-O-methyl-3-oxoerythronolide A, 11,12-carbamate;
19-Deoxo-1-deoxy-5-O-desosaminyl-11-(3-(3-(4-methoxyphenyl)-(1,2,4)oxadizol-5-yl)-propyl)hydrazo-9-methoxyimino-6-O-methyl-3-oxoerythronolide A, 11,12-carbamate;
9-Deoxo-11-deoxy-5-O-desosaminyl-11-(3-(3-(4-pyridin-4-yl)-(1,2,4)oxadizol-5-yl)-propyl)hydrazo-9-hydroxyimino-6-O-methyl-3-oxoerythronolide A, 11,12-carbamate;
9-Deoxo-11-deoxy-5-O-desosaminyl-11-(3-(3-(4-pyridin-4-yl)-(1,2,4)oxadizol-5-yl)-propyl)hydrazo-9-methoxyimino-6-O-methyl-3-oxoerythronolide A, 11,12-carbamate;
9-Deoxo-11-deoxy-5-O-desosaminyl-11-(3-naphthalen-1-yl-propyl)hydrazo-9-hydroxyimino-6-O-methyl-3-oxoerythronolide A, 11,12-carbamate;
9-Deoxo-11-deoxy-5-O-desosaminyl-11-(3-naphthalen-1-yl-propyl)hydrazo-9-methoxyimino-6-O-methyl-3-oxoerythronolide A, 11,12-carbamate;
9-Deoxo-11-deoxy-5-O-desosaminyl-11-(3-benzotrizol-1-yl-propyl)hydrazo-9-hydroxyimino-6-O-methyl-3-oxoerythronolide A, 11,12-carbamate;
9-Deoxo-11-deoxy-5-O-desosaminyl-11-(3-benzotrizol-1-yl-propyl)hydrazo-9-methoxyimino-6-O-methyl-3-oxoerythronolide A, 11,12-carbamate;
9-Deoxo-11-deoxy-5-O-desosaminyl-11-(3-benzotrizol-2-yl-propyl)hydrazo-9-hydroxyimino-6-O-methyl-3-oxoerythronolide A, 11,12-carbamate;
9-Deoxo-11-deoxy-5-O-desosaminyl-11-(3-benzotrizol-2-yl-propyl)hydrazo-9-methoxyimino-6-O-methyl-3-oxoerythronolide A, 11,12-carbamate;
9-Deoxo-11-deoxy-5-O-desosaminyl-11-(3-(1H-indol-3-yl)-propyl)hydrazo-9-hydroxyimino-6-O-methyl-3-oxoerythronolide A, 11,12-carbamate;
9-Deoxo-11-deoxy-5-O-desosaminyl-11-(3-(1H-indol-3-yl)-propyl)hydrazo-9-methoxyimino-6-O-methyl-3-oxoerythronolide A, 11,12-carbamate;
9-Deoxo-11-deoxy-5-O-desosaminyl-11-(3-pyridin-4-yl-propyl)hydrazo-9-hydroxyimino-6-O-methyl-3-oxoerythronolide A, 11,12-carbamate;
9-Deoxo-11-deoxy-5-O-desosaminyl-11-(3-pyridin-4-yl-propyl)hydrazo-9-methoxyimino-6-O-methyl-3-oxoerythronolide A, 11,12-carbamate;
9-Deoxo-11-deoxy-5-O-desosaminyl-11-(3-pyridin-3-yl-propyl)hydrazo-9-hydroxyimino-6-O-methyl-3-oxoerythronolide A, 11,12-carbamate;
9-Deoxo-11-deoxy-5-O-desosaminyl-11-(3-pyridin-3-yl-propyl)hydrazo-9-methoxyimino-6-O-methyl-3-oxoerythronolide A, 11,12-carbamate;
9-Deoxo-11-deoxy-5-O-desosaminyl-11-(3-(pyridin-2-yl-propyl)hydrazo-9-hydroxyimino-6-O-methyl-3-oxoerythronolide A, 11,12-carbamate;
9-Deoxo-11-deoxy-5-O-desosaminyl-11-(3-(pyridin-2-yl-propyl)hydrazo-9-methoxyimino-6-O-methyl-3-oxoerythronolide A, 11,12-carbamate;
9-Deoxo-11-deoxy-5-O-desosaminyl-11-(3-phenylpropyl)hydrazo-9-hydroxyimino-6-O-methyl-3-oxoerythronolide A, 11,12-carbamate;
9-Deoxo-11-deoxy-5-O-desosaminyl-11-(3-phenylpropyl)hydrazo-9-methoxyimino-6-O-methyl-3-oxoerythronolide A, 11,12-carbamate;
9-Deoxo-11-deoxy-5-O-desosaminyl-11-bis-(3-phenylpropyl)hydrazo-9-hydroxyimino-6-O-methyl-3-oxoerythronolide A, 11,12-carbamate;
9-Deoxo-11-deoxy-5-O-desosaminyl-11-bis-(3-phenylpropyl)hydrazo-9-methoxyimino-6-O-methyl-3-oxoerythronolide A, 11,12-carbamate;
9-Deoxo-11-deoxy-5-O-desosaminyl-11-(3-(4-methoxyphenyl)-propyl)hydrazo-9-hydroxyimino-6-O-methyl-3-oxoerythronolide A, 11,12-carbamate;
9-Deoxo-11-deoxy-5-O-desosaminyl-11-(3-(4-methoxyphenyl)-propyl)hydrazo-9-methoxyimino-6-O-methyl-3-oxoerythronolide A, 11,12-carbamate;
9-Deoxo-11-deoxy-5-O-desosaminyl-11-(3-(3-methoxyphenyl)-propyl)hydrazo-9-hydroxyimino-6-O-methyl-3-oxoerythronolide A, 11,12-carbamate;
9-Deoxo-11-deoxy-5-O-desosaminyl-11-(3-(3-methoxyphenyl)-propyl)hydrazo-9-methoxyimino-6-O-methyl-3-oxoerythronolide A, 11,12-carbamate;
9-Deoxo-11-deoxy-5-O-desosaminyl-11-(3-(2-methoxyphenyl)-propyl)hydrazo-9-hydroxyimino-6-O-methyl-3-oxoerythronolide A, 11,12-carbamate;
9-Deoxo-11-deoxy-5-O-desosaminyl-11-(3-(2-methoxyphenyl)-propyl)hydrazo-9-methoxyimino-6-O-methyl-3-oxoerythronolide A, 11,12-carbamate;
9-Deoxo-11-deoxy-5-O-desosaminyl-11-(3-(4-hydroxyphenyl)-propyl)hydrazo-9-hydroxyimino-6-O-methyl-3-oxoerythronolide A, 11,12-carbamate;
9-Deoxo-11-deoxy-5-O-desosaminyl-11-(3-(4-hydroxyphenyl)-propyl)hydrazo-9-methoxyimino-6-O-methyl-3-oxoerythronolide A, 11,12-carbamate;
9-Deoxo-11-deoxy-5-O-desosaminyl-11-(3-(3-methoxyphenyl)-propyl)hydrazo-9-hydroxyimino-6-O-methyl-3-oxoerythronolide A, 11,12-carbamate;
9-Deoxo-11-deoxy-5-O-desosaminyl-11-(3-(3-methoxyphenyl)-propyl)hydrazo-9-methoxyimino-6-O-methyl-3-oxoerythronolide A, 11,12-carbamate;
9-Deoxo-11-deoxy-5-O-desosaminyl-11-(2-phenylethyl)hydrazo-9-hydroxyimino-6-O-methyl-3-oxoerythronolide A, 11,12-carbamate;
9-Deoxo-11-deoxy-5-O-desosaminyl-11-(2-phenylethyl)hydrazo-9-methoxyimino-6-O-methyl-3-oxoerythronolide A, 11,12-carbamate;
9-Deoxo-11-deoxy-5-O-desosaminyl-11-(4-phenylbutyl)hydrazo-9-hydroxyimino-6-O-methyl-3-oxoerythronolide A, 11,12-carbamate;
9-Deoxo-11-deoxy-5-O-desosaminyl-11-(4-phenylbutyl)hydrazo-9-methoxyimino-6-O-methyl-3-oxoerythronolide A, 11,12-carbamate;
9-Deoxo-11-deoxy-5-O-desosaminyl-11-(3-furan-2-yl-propyl)hydrazo-9-hydroxyimino-6-O-methyl-3-oxoerythronolide A, 11,12-carbamate;
9-Deoxo-11-deoxy-5-O-desosaminyl-11-(3-furan-2-yl-propyl)hydrazo-9-methoxyimino-6-O-methyl-3-oxoerythronolide A, 11,12-carbamate;
9-Deoxo-11-deoxy-5-O-desosaminyl-11-(3-thiophen-2-yl-propyl)hydrazo-9-hydroxyimino-6-O-methyl-3-oxoerythronolide A, 11,12-carbamate;
9-Deoxo-11-deoxy-5-O-desosaminyl-11-(3-thiophen-2-yl-propyl)hydrazo-9-methoxyimino-6-O-methyl-3-oxoerythronolide A, 11,12-carbamate;
9-Deoxo-11-deoxy-5-O-desosaminyl-11-(3-pyrrol-1-yl-propyl)hydrazo-9-hydroxyimino-6-O-methyl-3-oxoerythronolide A, 11,12-carbamate;
9-Deoxo-11-deoxy-5-O-desosaminyl-11-(3-pyrrol-1-yl-propyl)hydrazo-9-methoxyimino-6-O-methyl-3-oxoerythronolide A, 11,12-carbamate;
9-Deoxo-11-deoxy-5-O-desosaminyl-11-(3-pyrazol-1-yl-propyl)hydrazo-9-hydroxyimino-6-O-methyl-3-oxoerythronolide A, 11,12-carbamate;
9-Deoxo-11-deoxy-5-O-desosaminyl-11-(3-pyrazol-1-yl-propyl)hydrazo-9-methoxyimino-6-O-methyl-3-oxoerythronolide A, 11,12-carbamate;
9-Deoxo-11-deoxy-5-O-desosaminyl-11-(3-(2-pyridin-3-yl-thiazol-4-yl)-propyl)hydrazo-9-hydroxyimino-6-O-methyl-3-oxoerythronolide A, 11,12-carbamate;
9-Deoxo-11-deoxy-5-O-desosaminyl-11-(3-(2-pyridin-3-yl-thiazol-4-yl)-propyl)hydrazo-9-methoxyimino-6-O-methyl-3-oxoerythronolide A, 11,12-carbamate;
9-Deoxo-11-deoxy-5-O-desosaminyl-11-(3-(2-phenyl-thiazol-5-yl)-propyl)hydrazo-9-hydroxyimino-6-O-methyl-3-oxoerythronolide A, 11,12-carbamate;
9-Deoxo-11-deoxy-5-O-desosaminyl-11-(3-(2-phenyl-thiazol-5-yl)-propyl)hydrazo-9-methoxyimino-6-O-methyl-3-oxoerythronolide A, 11,12-carbamate;
9-Deoxo-11-deoxy-5-O-desosaminyl-11-(3-(4-phenyl-1H-imidazol-2-yl)-propyl)hydrazo-9-hydroxyimino-6-O-methyl-3-oxoerythronolide A, 11,12-carbamate;
9-Deoxo-11-deoxy-5-O-desosaminyl-11-(3-(4-phenyl-1H-imidazol-2-yl)-propyl)hydrazo-9-methoxyimino-6-O-methyl-3-oxoerythronolide A, 11,12-carbamate;
9-Deoxo-11-deoxy-5-O-desosaminyl-10-epi-11-hydrazo-9-benzoxyimino-6-O-methyl-3-oxoerythronolide A, 11,12-carbamate;
9-Deoxo-11-deoxy-5-O-desosaminyl-10-epi-11-hydrazo-9-hydroxyimino-6-O-methyl-3-oxoerythronolide A, 11,12-carbamate; and the pharmaceutically acceptable salts of the foregoing compounds.

10. A pharmaceutical composition for the treatment of a bacterial infection or a protozoa infection in a mammal, fish or bird which comprises a therapeutically effective amount of a compound of claim 1 and a pharmaceutically acceptable carrier.

11. A method of treating a bacterial infection or a protozoa infection in a mammal, fish, or bird which comprises administering to said mammal, fish, or bird a therapeutically effective amount of a compound of claim 1.

12. A method of preparing a compound of the formula 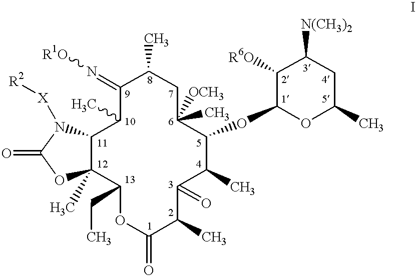
or a pharmaceutically acceptable salt thereof, wherein:
X is —CR⁷R⁸— or —NR⁷—;
or X is taken together with R² to form —N═CR⁴R⁵;
or X and R² are taken together to form a heterocyclic ring of the formula XVI: 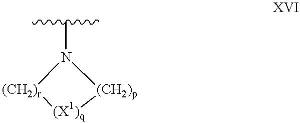
wherein in said ring of formula XVI, r and p are each independently an integer ranging from 1 to 3, q is 0 or 1, and X¹ is —CH₂—, O, S, —C(O)—, —C(S)—, —SO₂—, —CH═CH—, —CH(OH)CH(OH)—, or —NH—; and wherein the (CH₂)ᵣ and (CH₂)ₚ portions of said ring of formula XVI are optionally substituted by 1 to 4 substituents, and the nitrogen atom where X¹ is —NH— is optionally substituted by 1 substituent, said optional substituents being independently selected from the group consisting of —C(O)O(C₁-C₁₀ alkyl), C₁-C₁₀ alkoxy, C₁-C₁₀ alkanoyl, halo, nitro, cyano, 5-10 membered heterocyclyl, C₁-C₁₀ alkyl, —NR⁷R⁸, C₆-C₁₀ aryl, —S(O)ₙ(C₁-C₁₀ alkyl) wherein n is an integer ranging from 0 to 2, and —SO₂NR⁷R⁸;
R¹ is H or C₁-C₁₀ alkyl, wherein 1 to 3 carbons of said alkyl are optionally replaced by a heteroatom selected from O, S and N, and said alkyl is optionally substituted by 1 to 3 substituents independently selected from the group consisting of —C(O)O(C₁-C₁₀ alkyl), C₁-C₁₀ alkoxy, C₁-C₁₀ alkanoyl, halo, nitro, cyano, 5-10 membered heterocyclyl, C₁-C₁₀ alkyl, —NR⁷R⁸, C₆-C₁₀ aryl, —S(O)ₙ(C₁-C₁₀ alkyl) wherein n is an integer ranging from 0 to 2, and —SO₂NR⁷R⁸;
R² is (i) H, R⁴, —C(O)R⁴, —C(O)OR⁴ or —(CR⁷R⁸)ₘR³ when X is —NR⁷—, or (ii) H, R⁴, or —(CR⁷R⁸)ₘR³ when X is —CR⁷R⁸—, wherein for both (i) and (ii) m is an integer ranging from 0 to 6 and both R⁷ and R⁸ may vary where m is greater than 1;
each R³ is independently C₆-C₁₀ aryl or 5-10 membered heterocyclyl, wherein said aryl and heterocyclyl groups are optionally substituted by 1 to 3 substituents independently selected from the group consisting of —C(O)O(C₁-C₁₀ alkyl), C₁-C₁₀ alkoxy, C₁-C₁₀ alkanoyl, halo, nitro, cyano, 5-10 membered heterocyclyl, C₆-C₁₀ aryl, C₁-C₁₀ alkyl, —NR⁷R⁸, —S(O)ₙ(C₁-C₁₀ alkyl) wherein n is an integer ranging from 0 to 2, and —SO₂NR⁷R⁸; and,
each R⁴ and R⁵ is independently selected from H and C₁-C₁₂ alkyl wherein one or two carbons of said alkyl are optionally replaced be a heteroatom selected from O, S and N, and wherein said alkyl is optionally substituted by 1 to 3 substituents independently selected from the group consisting of —C(O)O(C₁-C₁₀ alkyl), C₁-C₁₀ alkoxy, C₁-C₁₀ alkanoyl, halo, nitro, cyano, C₁-C₁₀ alkyl, —NR⁷R⁸, C₆-C₁₀ aryl, 5-10 membered heterocyclyl, —S(O)ₙ(C₁-C₁₀ alkyl) wherein n is an integer ranging from 0 to 2, and —SO₂NR⁷R⁸;
R⁶ is H, —C(O)R³ or C₁-C₁₈ alkanoyl, wherein in the alkyl portion of said alkanoyl one or two carbons optionally may be replaced by a heteroatom selected from O, S and N; and,
each R⁷ and R⁸ is independently H or C₁-C₆ alkyl;
which comprises treating a compound of the formula 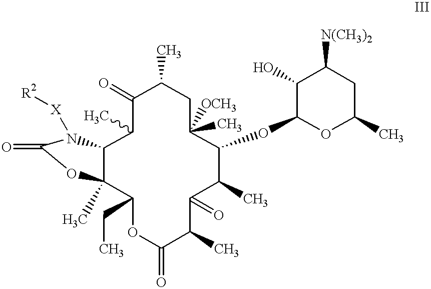
wherein X and R² are as defined for said compound of formula I, with a compound of the formula R¹ONH₂.HCl or R¹ONH₂, wherein R¹ is as defined for said compound of formula I, in the presence of an acid in a polar solvent.

13. The process of claim 12 wherein said solvent is methanol, ethanol, or isopropyl alcohol.

14. The process of claim 12 wherein said acid is Py.HCl, wherein Py is pyridine, or Et₃N.HCl wherein Et is ethyl.
